(19) United States
(12) Patent Application Publication
(10) Pub. No.: US 2005/0222064 A1
(43) Pub. Date: Oct. 6, 2005

(54) Polycationic compositions for cellular delivery of polynucleotides

(75) Inventors: Chandra Vargeese, Broomfield, CO (US); Weimin Wang, Superior, CO (US); Tongqian Chen, Longmont, CO (US); David Sweedler, Louisville, CO (US); Peter Haeberli, Berthoud, CO (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Sirna Therapeutics, Inc., Boulder, CO (US)

(21) Appl. No.: 10/888,269

(22) Filed: Jul. 9, 2004

Related U.S. Application Data
(63) Continuation-in-part of application No. PCT/US04/16390, filed on May 24, 2004.
Application No. PCT/US04/16390 is a continuation-in-part of application No. 10/826,966, filed on Apr. 16, 2004.
Application No. 10/826,966 is a continuation-in-part of application No. 10/757,803, filed on Jan. 14, 2004.
Application No. 10/757,803 is a continuation-in-part of application No. 10/720,448, filed on Nov. 24, 2003.
Application No. 10/720,448 is a continuation-in-part of application No. 10/693,059, filed on Oct. 23, 2003.
Application No. 10/693,059 is a continuation-in-part of application No. 10/444,853, filed on May 23, 2003.
Application No. 10/444,853 is a continuation-in-part of application No. PCT/US03/05346, filed on Feb. 20, 2003.
Application No. 10/444,853 is a continuation-in-part of application No. PCT/US03/05028, filed on Feb. 20, 2003.
(60) Provisional application No. 60/485,667, filed on Jul. 9, 2003.
(60) Provisional application No. 60/358,580, filed on Feb. 20, 2002.
(60) Provisional application No. 60/363,124, filed on Mar. 11, 2002.
(60) Provisional application No. 60/386,782, filed on Jun. 6, 2002.
(60) Provisional application No. 60/406,784, filed on Aug. 29, 2002.
(60) Provisional application No. 60/408,378, filed on Sep. 5, 2002.
(60) Provisional application No. 60/409,293, filed on Sep. 9, 2002.
(60) Provisional application No. 60/440,129, filed on Jan. 15, 2003.

(51) Int. Cl. A61K 48/00; A61K 31/4172; A61K 31/155

(52) U.S. Cl. 514/44; 514/400; 514/634

(57) ABSTRACT

The present invention relates to delivery of biologically active molecules to cells. Specifically, the invention relates to polycationic compositions, polymers and methods for delivering nucleic acids, polynucleotides, and oligonucleotides such RNA, DNA and analogs thereof, including short interfering RNA (siRNA), ribozymes, and antisense, or peptides, polypeptides, proteins, antibodies, hormones and small molecules, to cells by facilitating transport across cellular membranes epithelial tissues and endothelial tissues. The compositions and methods of the invention are useful in therapeutic, research, and diagnostic applications that rely upon the efficient transfer of biologically active molecules into cells, tissues, and organs.

This application claims the benefit of U.S. Provisional Application No. 60/485,667 filed Jul. 9, 2003. This application is also a continuation-in-part of International Patent Application No. PCT/US04/16390, filed May 24, 2004, which is a continuation-in-part of U.S. patent application Ser. No. 10/826,966, filed Apr. 16, 2004, which is continuation-in-part of U.S. patent application Ser. No. 10/757,803, filed Jan. 14, 2004, which is a continuation-in-part of U.S. patent application Ser. No. 10/720,448, filed Nov. 24, 2003, which is a continuation-in-part of U.S. patent application Ser. No. 10/693,059, filed Oct. 23, 2003, which is a continuation-in-part of U.S. patent application Ser. No. 10/444,853, filed May 23, 2003, which is a continuation-in-part of International Patent Application No. PCT/US03/05346, filed Feb. 20, 2003, and a continuation-in-part of International Patent Application No. PCT/US03/05028, filed Feb. 20, 2003, both of which claim the benefit of U.S. Provisional Application No. 60/358,580 filed Feb. 20, 2002, U.S. Provisional Application No. 60/363,124 filed Mar. 11, 2002, U.S. Provisional Application No. 60/386,782 filed Jun. 6, 2002, U.S. Provisional Application No. 60/406,784 filed Aug. 29, 2002, U.S. Provisional Application No. 60/408,378 filed Sep. 5, 2002, U.S. Provisional Application No. 60/409,293 filed Sep. 9, 2002, and U.S. Provisional Application No. 60/440,129 filed Jan. 15, 2003. The instant application claims priority to all of the listed applications, which are hereby incorporated by reference herein in their entireties, including the drawings.

### BACKGROUND OF THE INVENTION

The present invention relates to the delivery of biologically active molecules to cells. Specifically, the invention relates to compounds, compositions and methods for delivering nucleic acids, polynucleotides, and oligonucleotides such RNA, DNA and analogs thereof, peptides, polypeptides, proteins, antibodies, hormones and small molecules, to cells by facilitating transport across cellular membranes epithelial tissues and endothelial tissues. The compounds, compositions and methods of the invention are useful in therapeutic, research, and diagnostic applications that rely upon the efficient transfer of biologically active molecules into cells, tissues, and organs. The discussion is provided only for understanding of the invention that follows. This summary is not an admission that any of the work described below is prior art to the claimed invention.

The cellular delivery of various therapeutic compounds, such as antiviral and chemotherapeutic agents, is usually compromised by two limitations. First the selectivity of a number of therapeutic agents is often low, resulting in high toxicity to normal tissues. Secondly, the trafficking of many compounds into living cells is highly restricted by the complex membrane systems of the cell. Specific transporters allow the selective entry of nutrients or regulatory molecules, while excluding most exogenous molecules such as nucleic acids and proteins. Various strategies can be used to improve transport of compounds into cells, including the use of lipid carriers, biodegradable polymers, and various conjugate systems.

The most well studied approaches for improving the transport of foreign nucleic acids into cells involve the use of viral vectors or cationic lipids and related cytofectins. Viral vectors can be used to transfer genes efficiently into some cell types, but they generally cannot be used to introduce chemically synthesized molecules into cells. An alternative approach is to use delivery formulations incorporating cationic lipids, which interact with nucleic acids through one end and lipids or membrane systems through another (for a review see Felgner, 1990, Advanced Drug Delivery Reviews, 5, 162-187; Felgner 1993, J. Liposome Res., 3, 3-16). Synthetic nucleic acids as well as plasmids can be delivered using the cytofectins, although the utility of such compounds is often limited by cell-type specificity, requirement for low serum during transfection, and toxicity.

Since the first description of liposomes in 1965, by Bangham (J. Mol. Biol. 13, 238-252), there has been a sustained interest and effort in the area of developing lipid-based carrier systems for the delivery of pharmaceutically active compounds. Liposomes are attractive drug carriers since they protect biological molecules from degradation while improving their cellular uptake. One of the most commonly used classes of liposome formulations for delivering polyanions (e.g., DNA) is that which contains cationic lipids. Lipid aggregates can be formed with macromolecules using cationic lipids alone or including other lipids and amphiphiles such as phosphatidylethanolamine. It is well known in the art that both the composition of the lipid formulation as well as its method of preparation have effect on the structure and size of the resultant anionic macromolecule-cationic lipid aggregate. These factors can be modulated to optimize delivery of polyanions to specific cell types in vitro and in vivo. The use of cationic lipids for cellular delivery of biologically active molecules has several advantages. The encapsulation of anionic compounds using cationic lipids is essentially quantitative due to electrostatic interaction. In addition, it is believed that the cationic lipids interact with the negatively charged cell membranes initiating cellular membrane transport (Akhtar et al., 1992, Trends Cell Bio., 2, 139; Xu et al., 1996, Biochemistry 35, 5616).

Another approach to delivering biologically active molecules involves the use of conjugates. Conjugates are often selected based on the ability of certain molecules to be selectively transported into specific cells, for example via receptor-mediated endocytosis. By attaching a compound of interest to molecules that are actively transported across the cellular membranes, the effective transfer of that compound into cells or specific cellular organelles can be realized. Alternately, molecules that are able to penetrate cellular membranes without active transport mechanisms, for example, various lipophilic molecules, can be used to deliver compounds of interest. Examples of molecules that can be utilized as conjugates include but are not limited to peptides, hormones, fatty acids, vitamins, flavonoids, sugars, reporter molecules, reporter enzymes, chelators, porphyrins, intercalcators, and other molecules that are capable of penetrating cellular membranes, either by active transport or passive transport.

The delivery of compounds to specific cell types, for example, cancer cells or cells specific to particular tissues and organs, can be accomplished by utilizing receptors associated with specific cell types. Particular receptors are overexpressed in certain cancerous cells, including the high affinity folic acid receptor. For example, the high affinity folate receptor is a tumor marker that is overexpressed in a variety of neoplastic tissues, including breast, ovarian, cervical, colorectal, renal, and nasoparyngeal tumors, but is expressed to a very limited extent in normal tissues. The use of folic acid based conjugates to transport exogenous compounds across cell membranes can provide a targeted delivery approach to the treatment and diagnosis of disease and can provide a reduction in the required dose of therapeutic compounds. Furthermore, therapeutic bioavailability, pharmacodynamics, and pharmacokinetic parameters can be modulated through the use of bioconjugates, including folate bioconjugates. Godwin et al., 1972, J. Biol. Chem., 247, 2266-2271, report the synthesis of biologically active pteroyloligo-L-glutamates. Habus et al., 1998, Bioconjugate Chem., 9, 283-291, describe a method for the solid phase synthesis of certain oligonucleotide-folate conjugates. Cook, U.S. Pat. No. 6,721,208, describes certain oligonucleotides modified with specific conjugate groups. The use of biotin and folate conjugates to enhance transmembrane transport of exogenous molecules, including specific oligonucleotides has been reported by Low et al., U.S. Pat. Nos. 5,416,016, 5,108,921, and International PCT publication No. WO 90/12096. Manoharan et al., International PCT publication No. WO 99/66063 describe certain folate conjugates, including specific nucleic acid folate conjugates with a phosphoramidite moiety attached to the nucleic acid component of the conjugate, and methods for the synthesis of these folate conjugates. Nomura et al., 2000, J. Org. Chem., 65, 5016-5021, describe the synthesis of an intermediate, alpha-[2-(trimethylsilyl)ethoxycarbonyl]folic acid, useful in the synthesis of ceratin types of folate-nucleoside conjugates. Guzaev et al., U.S. Pat. No. 6,335,434, describes the synthesis of certain folate oligonucleotide conjugates.

The delivery of compounds to other cell types can be accomplished by utilizing receptors associated with a certain type of cell, such as hepatocytes. For example, drug delivery systems utilizing receptor-mediated endocytosis have been employed to achieve drug targeting as well as drug-uptake enhancement. The asialoglycoprotein receptor (ASGPr) (see for example Wu and Wu, 1987, J. Biol. Chem. 262, 4429-4432) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose and galactosamine based conjugates to transport exogenous compounds across cell membranes can provide a targeted delivery approach to the treatment of liver disease such as HBV and HCV infection or hepatocellular carcinoma. The use of bioconjugates can also provide a reduction in the required dose of therapeutic compounds required for treatment. Furthermore, therapeutic bioavailability, pharmacodynamics, and pharmacokinetic parameters can be modulated through the use of bioconjugates.

A number of peptide based cellular transporters have been developed by several research groups. These peptides are capable of crossing cellular membranes in vitro and in vivo with high efficiency. Examples of such fusogenic peptides include a 16-amino acid fragment of the homeodomain of ANTENNAPEDIA, a Drosophila transcription factor (Wang et al., 1995, PNAS USA., 92, 3318-3322); a 17-mer fragment representing the hydrophobic region of the signal sequence of Kaposi fibroblast growth factor with or without NLS domain (Antopolsky et al., 1999, Bioconj. Chem., 10, 598-606); a 17-mer signal peptide sequence of caiman crocodylus Ig(5) light chain (Chaloin et al., 1997, Biochem. Biophys. Res. Comm., 243, 601-608); a 17-amino acid fusion sequence of HIV envelope glycoprotein gp4114, (Morris et al., 1997, Nucleic Acids Res., 25, 2730-2736); the HIV-1 Tat49-57 fragment (Schwarze et al., 1999, Science, 285, 1569-1572); a transportan A-achimeric 27-mer consisting of N-terminal fragment of neuropeptide galanine and membrane interacting wasp venom peptide mastoporan (Lindgren et al., 2000, Bioconjugate Chem., 11, 619-626); and a 24-mer derived from influenza virus hemagglutinin envelop glycoprotein (Bongartz et al., 1994, Nucleic Acids Res., 22, 4681-4688).

These peptides were successfully used as part of an antisense oligodeoxyribonucleotide-peptide conjugate for cell culture transfection without lipids. In a number of cases, such conjugates demonstrated better cell culture efficacy then parent oligonucleotides transfected using lipid delivery. In addition, use of phage display techniques has identified several organ targeting and tumor targeting peptides in vivo (Ruoslahti, 1996, Ann. Rev. Cell Dev. Biol., 12, 697-715). Conjugation of tumor targeting peptides to doxorubicin has been shown to significantly improve the toxicity profile and has demonstrated enhanced efficacy of doxorubicin in the in vivo murine cancer model MDA-MB-435 breast carcinoma (Arap et al., 1998, Science, 279, 377-380).

Another approach to the intracellular delivery of biologically active molecules involves the use of cationic polymers. For example, Ryser et al., International PCT Publication No. WO 79/00515 describes the use of high molecular weight lysine polymers for increasing the transport of various molecules across cellular membranes. Rothbard et al., International PCT Publication No. WO 98/52614, describes certain methods and compositions for transporting drugs and macromolecules across biological membranes in which the drug or macromolecule is covalently attached to a transport polymer consisting of from 6 to 25 subunits, at least 50% of which contain a guanidino or amidino side chain. The transport polymers are preferably polyarginine peptides composed of all D-, all L- or mixtures of D- and L-arginine. Rothbard et al., U.S. Patent Application Publication No. 20030082356, describes certain poly-lysine and poly-arginine compounds for the delivery of drugs and other agents across epithelial tissues, including the skin, gastrointestinal tract, pulmonary epithelium and blood brain barrier. Wendel et al., U.S. Patent Application Publication No. 20030032593, describes certain polyarginine compounds. Rothbard et al., U.S. Patent Application Publication No. 20030022831, describes certain poly-lysine and poly-arginine compounds for intra-ocular delivery of drugs. Kosak, U.S. Patent Application Publication No. 20010034333, describes certain cyclodextran polymers compositions that include a cross-linked cationic polymer component. Lewis et al., U.S. Patent Application Publication No. 20030125281, describes certain compositions consisting of the combination of siRNA, certain amphipathic compounds, and certain polycations.

### SUMMARY OF THE INVENTION

The present invention features compounds, compositions, and methods to facilitate delivery of molecules into a biological system, such as cells. The compounds, compositions, and methods provided by the instant invention can impart therapeutic activity by transferring therapeutic compounds across cellular membranes or across one or more layers of epithelial or endothelial tissue. The present invention encompasses the design and synthesis of novel agents for the delivery of molecules, including but not limited to small molecules, lipids, nucleosides, nucleotides, nucleic acids, polynucleotides, oligonucleotides, antibodies, toxins, negatively charged polymers and other polymers, for example proteins, peptides, hormones, carbohydrates, or polyamines, across cellular membranes. Non-limiting examples of polynucleotides that can be delivered across cellular membranes using the compounds and methods of the invention include short interfering nucleic acid (siNA), antisense, enzymatic nucleic acid molecules, 2′,5′-oligoadenylate, triplex forming oligonucleotides, aptamers, and decoys. In general, the transporters described are designed to be used either individually or as part of a multi-component system, with or without degradable linkers. The compounds of the invention generally shown in the Formulae below, when formulated into compositions, are expected to improve delivery of molecules into a number of cell types originating from different tissues, in the presence or absence of serum.

The compounds, compositions, and methods of the invention are useful for delivering biologically active molecules (e.g. nucleic acids, polynucleotides, oligonucleotides, peptides, polypeptides, proteins, hormones, antibodies, and small molecules) to cells or across epithelial and endothelial tissues, such as skin, mucous membranes, vasculature tissues, gastrointestinal tissues, blood brain barrier tissues, opthamological tissues, pulmonary tissues, liver tissues, cardiac tissues, kidney tissues etc.). The compounds, compositions, and methods of the invention can be used both for delivery to a particular site of administration or for systemic delivery.

The compounds, compositions, and methods of the invention can increase delivery or availability of biologically active molecules (e.g. nucleic acids, poly nucleotides, oligonucleotides, peptides, polypeptides, proteins, hormones, antibodies, and small molecules) to cells or tissues compared to delivery of the molecules in the absence of the compounds, compositions, and methods of the invention. As such, the level of a biologically active molecule inside a cell, tissue, or organism is increased in the presence of the compounds and compositions of the invention compared to when the compounds and compositions of the invention are absent.

The present invention features a compound having the Formula 1: 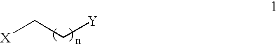
wherein X and Y are the same or different and represent an amine, substituted amine, guanidinium, substituted guanidinium, histidyl, or substituted histidyl group, and n represents an integer from 0 to about 20. A non-limiting example of a compound having Formula 1 is CAS Registry No. 473759-22-7.

The present invention features a compound having the Formula 2: 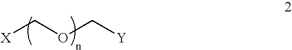
wherein X and Y are the same or different and represent an amine, substituted amine, guanidinium, substituted guanidinium, histidyl, or substituted histidyl group, and n represents an integer from about 1 to about 20.

The present invention features a compound having the Formula 3: 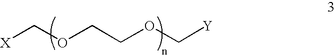
wherein X and Y are the same or different and represent an amine, substituted amine, guanidinium, substituted guanidinium, histidyl, or substituted histidyl group, and n represents an integer from about 1 to about 20.

The present invention features a compound having the Formula 4: 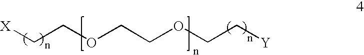
wherein X and Y are the same or different and represent an amine, substituted amine, guanidinium, substituted guanidinium, histidyl, or substituted histidyl group and each n independently represents an integer from about 1 to about 20.

The present invention features a compound having the Formula 5: 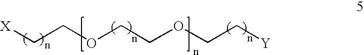
wherein X and Y are the same or different and represent an amine, substituted amine, guanidinium, substituted guanidinium, histidyl, or substituted histidyl group, and each n independently represents an integer from about 1 to about 20.

The present invention features a compound having the Formula 6: 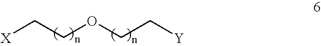
wherein X and Y are the same or different and represent an amine, substituted amine, guanidinium, substituted guanidinium, histidyl, or substituted histidyl group, and each n independently represents an integer from 0 to about 20.

The present invention features a compound having the Formula 7: 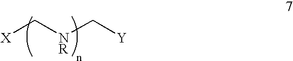
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and n represents an integer from about 1 to about 20.

The present invention features a compound having the Formula 8: 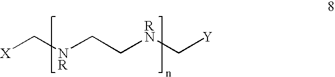
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and n represents an integer from about 1 to about 20.

The present invention features a compound having the Formula 9: 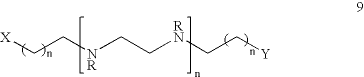
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and each n independently represents an integer from about 1 to about 20.

The present invention features a compound having the Formula 10: 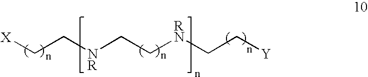
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and each n independently represents an integer from about 1 to about 20.

The present invention features a compound having the Formula 11: 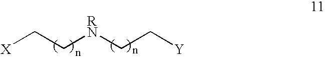
wherein X and Y are the same or different and represent an amine, substituted amine, guanidinium, substituted guanidinium, histidyl, or substituted histidyl group that can be the same or different, R represents H, alkyl, substituted alkyl, aryl, substituted aryl, or a ligand, and each n independently represents an integer from 0 to about 20.

The present invention features a compound having the Formula 12: 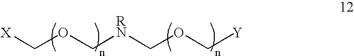
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and each n independently represents an integer from 0 to about 20.

The present invention features a compound having the Formula 13: 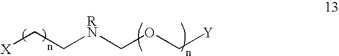
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and each n independently represents an integer from 0 to about 20.

The present invention features a compound having the Formula 14: 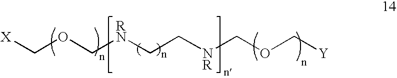
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and n′ represents an integer from about 1 to about 20.

The present invention features a compound having the Formula 15: 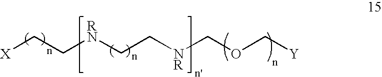
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and n′ represents an integer from about 1 to about 20.

The present invention features a compound having the Formula 16: 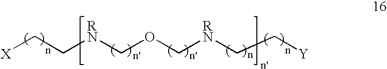
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and each n′ independently represents an integer from about 1 to about 10.

The present invention features a compound having the Formula 17: 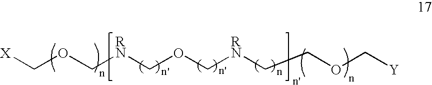
wherein X and Y are the same or different and represent an amine, substituted amine, guanidinium, substituted guanidinium, histidyl, or substituted histidyl group R represents H, alkyl, substituted alkyl, aryl, substituted aryl, or a ligand, each n independently represents an integer from 0 to about 20, and each n′ independently represents an integer from about 1 to about 10.

The present invention features a compound having the Formula 18: 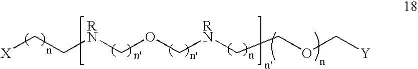
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and each n′ independently represents an integer from about 1 to about 10.

The present invention features a compound having the Formula 19: 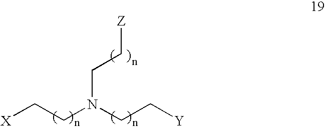
wherein X, Y and Z are the same or different and represent an amine, substituted amine, guanidinium, substituted guanidinium, histidyl, or substituted histidyl, and each n independently represents an integer from about 1 to about 20.

The present invention features a compound having the Formula 20: 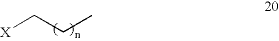
wherein X represents an amine, substituted amine, guanidinium, substituted guanidinium, histidyl, or substituted histidyl group, and n represents an integer from 0 to about 20.

The present invention features a compound having the Formula 21: 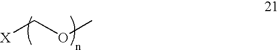
wherein X represents an amine, substituted amine, guanidinium, substituted guanidinium, histidyl, or substituted histidyl group, and n represents an integer from about 1 to about 20.

The present invention features a compound having the Formula 22: 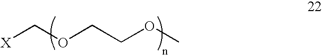
wherein X represents an amine, substituted amine, guanidinium, substituted guanidinium, histidyl, or substituted histidyl group, and n represents an integer from about 1 to about 20.

The present invention features a compound having the Formula 23: 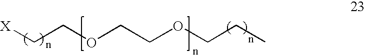
wherein X represents an amine, substituted amine, guanidinium, substituted guanidinium, histidyl, or substituted histidyl group, and each n independently represents an integer from about 1 to about 20.

The present invention features a compound having the Formula 24: 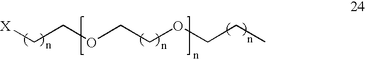
wherein X represents an amine, substituted amine, guanidinium, substituted guanidinium, histidyl, or substituted histidyl group, and each n independently represents an integer from about 1 to about 20.

The present invention features a compound having the Formula 25: 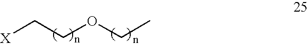
wherein X represents an amine, substituted amine, guanidinium, substituted guanidinium, histidyl, or substituted histidyl group, and each n independently represents an integer from 0 to about 20.

The present invention features a compound having the Formula 26: 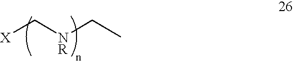
wherein X represents an amine, substituted amine, guanidinium, substituted guanidinium, histidyl, or substituted histidyl group, R represents H, alkyl, substituted alkyl, aryl, substituted aryl, or a ligand, and n represents an integer from about 1 to about 20.

The present invention features a compound having the Formula 27: 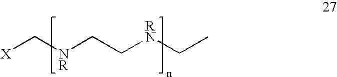
wherein X represents an amine, substituted amine, guanidinium, substituted guanidinium, histidyl, or substituted histidyl group, R represents H, alkyl, substituted alkyl, aryl, substituted aryl, or a ligand, and n represents an integer from about 1 to about 20.

The present invention features a compound having the Formula 28: 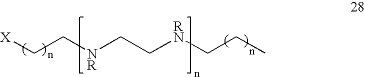
wherein X represents an amine, substituted amine, guanidinium, substituted guanidinium, histidyl, or substituted histidyl group, R represents H, alkyl, substituted alkyl, aryl, substituted aryl, or a ligand, and each n independently represents an integer from about 1 to about 20.

The present invention features a compound having the Formula 29: 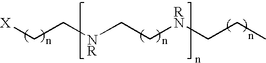
wherein X represents an amine, substituted amine, guanidinium, substituted guanidinium, histidyl, or substituted histidyl group, R represents H, alkyl, substituted alkyl, aryl, substituted aryl, or a ligand, and each n independently represents an integer from about 1 to about 20.

The present invention features a compound having the Formula 30: 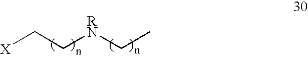
wherein X represents an amine, substituted amine, guanidinium, substituted guanidinium, histidyl, or substituted histidyl group, R represents H, alkyl, substituted alkyl, aryl, substituted aryl, or a ligand, and each n independently represents an integer from 0 to about 20.

The present invention features a compound having the Formula 31: 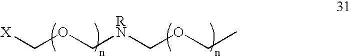
wherein X represents an amine, substituted amine, guanidinium, substituted guanidinium, histidyl, or substituted histidyl group, R represents H, alkyl, substituted alkyl, aryl, substituted aryl, or a ligand, and each n independently represents an integer from 0 to about 20.

The present invention features a compound having the Formula 32: 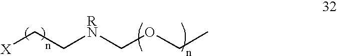
wherein X represents an amine, substituted amine, guanidinium, substituted guanidinium, histidyl, or substituted histidyl group, R represents H, alkyl, substituted alkyl, aryl, substituted aryl, or a ligand, and each n independently represents an integer from 0 to about 20.

The present invention features a compound having the Formula 33: 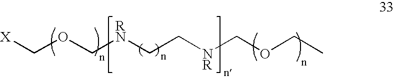
wherein X represents an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and n′ represents an integer from about 1 to about 20.

The present invention features a compound having the Formula 34: 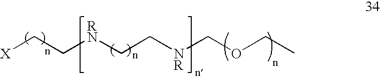
wherein X represents an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and n′ represents an integer from about 1 to about 20.

The present invention features a compound having the Formula 35: 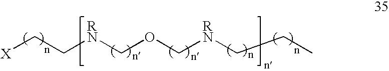
wherein X represents an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and each n′ independently represents an integer from about 1 to about 10.

The present invention features a compound having the Formula 36: 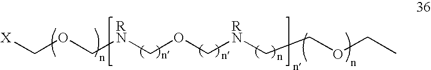
wherein X represents an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and each n′ independently represents an integer from about 1 to about 10.

The present invention features a compound having the Formula 37: 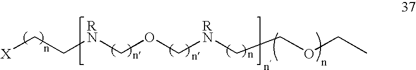
wherein X represents an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and each n′ independently represents an integer from about 1 to about 10.

The present invention features a compound having the Formula 38: 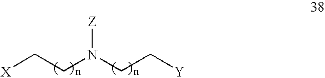
wherein X, and Y are the same or different and represent an amine, substituted amine, guanidinium, substituted guanidinium, histidyl, or substituted histidyl, Z represents a compound having any of Formulae 20-37, and each n independently represents an integer from about 1 to about 20.

The present invention features a compound having the Formula 39: 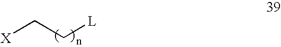
wherein X represents an amine, substituted amine, guanidinium, substituted guanidinium, histidyl, or substituted histidyl group, L represents a ligand, and n represents an integer from 0 to about 20.

The present invention features a compound having the Formula 40: 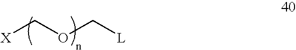
wherein X represents an amine, substituted amine, guanidinium, substituted guanidinium, histidyl, or substituted histidyl group, L represents a ligand, and n represents an integer from about 1 to about 20.

The present invention features a compound having the Formula 41: 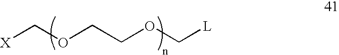
wherein X represents an amine, substituted amine, guanidinium, substituted guanidinium, histidyl, or substituted histidyl group, L represents a ligand, and n represents an integer from about 1 to about 20.

The present invention features a compound having the Formula 42: 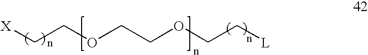
wherein X and Y each represent an amine, substituted amine, guanidinium, substituted guanidinium, histidyl, or substituted histidyl group that can be the same or different, and each n independently represents an integer from about 1 to about 20.

The present invention features a compound having the Formula 43: 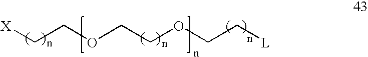
wherein X represents an amine, substituted amine, guanidinium, substituted guanidinium, histidyl, or substituted histidyl group, L represents a ligand, and each n independently represents an integer from about 1 to about 20.

The present invention features a compound having the Formula 44: 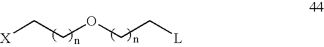
wherein X represents an amine, substituted amine, guanidinium, substituted guanidinium, histidyl, or substituted histidyl group, L represents a ligand, and each n independently represents an integer from 0 to about 20.

The present invention features a compound having the Formula 45: 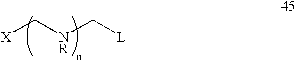
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, and n represents an integer from about 1 to about 20.

The present invention features a compound having the Formula 46: 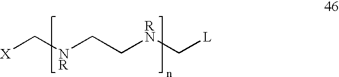
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, and n represents an integer from about 1 to about 20.

The present invention features a compound having the Formula 47: 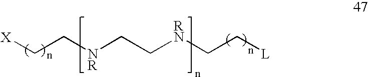
wherein X represents an amine, substituted amine, guanidinium, substituted guanidinium, histidyl, or substituted histidyl group, L represents a ligand, R represents 10H, alkyl, substituted alkyl, aryl, substituted aryl, or a ligand, and each n independently represents an integer from about 1 to about 20.

The present invention features a compound having the Formula 48: 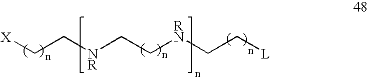
wherein X and Y each represent an amine, substituted amine, guanidinium, substituted guanidinium, histidyl, or substituted histidyl group that can be the same or different, R represents H, alkyl, substituted alkyl, aryl, substituted aryl, or a ligand, and each n independently represents an integer from about 1 to about 20.

The present invention features a compound having the Formula 49: 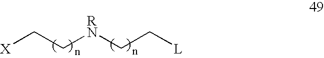
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, and each n independently represents an integer from 0 to about 20.

The present invention features a compound having the Formula 50: 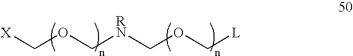
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, and each n independently represents an integer from 0 to about 20.

The present invention features a compound having the Formula 51: 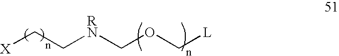
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, and each n independently represents an integer from 0 to about 20.

The present invention features a compound having the Formula 52: 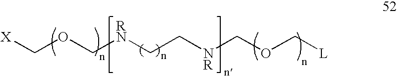
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, each n independently represents an integer from 0 to about 20, and n′ represents an integer from about 1 to about 20.

The present invention features a compound having the Formula 53: 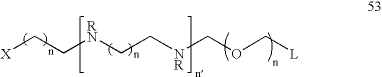
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, each n independently represents an integer from 0 to about 20, and n′ represents an integer from about 1 to about 20.

The present invention features a compound having the Formula 54: 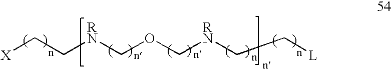
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, each n independently represents an integer from 0 to about 20, and each n′ independently represents an integer from about 1 to about 10.

The present invention features a compound having the Formula 55: 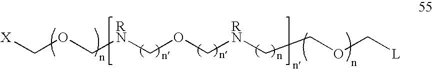
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, each n independently represents an integer from 0 to about 20, and each n′ independently represents an integer from about 1 to about 10.

The present invention features a compound having the Formula 56: 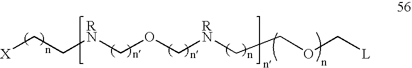
wherein X represents an amine, substituted amine, guanidinium, substituted guanidinium, histidyl, or substituted histidyl group, L represents a ligand, R represents H, alkyl, substituted alkyl, aryl, substituted aryl, or a ligand, each n independently represents an integer from 0 to about 20, and each n′ independently represents an integer from about 1 to about 10.

The present invention features a compound having the Formula 57: 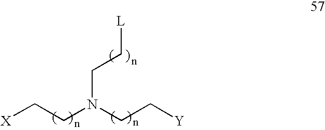
wherein X and Y are the same or different and represent an amine, substituted amine, guanidinium, substituted guanidinium, histidyl, or substituted histidyl group, L represents a ligand, and each n independently represents an integer from about 1 to about 20.

The present invention features a compound having the Formula 58: 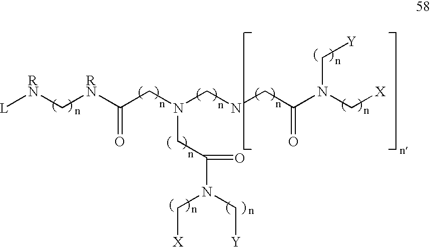
wherein X and Y are the same or different and represent an amine, substituted amine, guanidinium, substituted guanidinium, histidyl, or substituted histidyl group, L represents a ligand, R represents H, alkyl, substituted alkyl, aryl, substituted aryl, or a ligand, each n independently represents an integer from about 1 to about 20, and n′ is 1 or 2.

The present invention features a compound having the Formula 59: 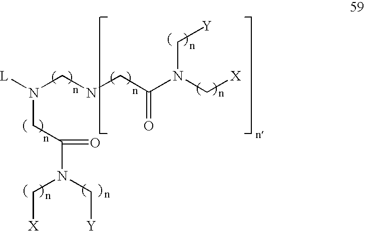
wherein X and Y are the same or different and represent an amine, substituted amine, guanidinium, substituted guanidinium, histidyl, or substituted histidyl group, L represents a ligand, each n independently represents an integer from about 1 to about 20, and n′ is 1 or 2.

The present invention features a compound having the Formula 60: 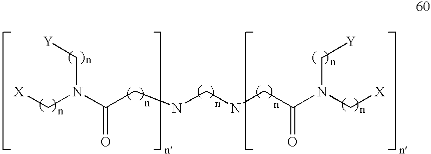
wherein X and Y are the same or different and represent an amine, substituted amine, guanidinium, substituted guanidinium, histidyl, or substituted histidyl group, each n independently represents an integer from about 1 to about 20, and each n′ is independently 1 or 2.

In one embodiment, the L of a compound having any of Formulae 39-57 comprises a ligand, for example a ligand that interacts with a receptor, such as a cell surface receptor, that allows the compound having any of Formulae 39-57 to interact with the receptor. Non-limiting examples of ligands include sugars and carbohydrates such as galactose, galactosamine, and N-acetyl galactosamine; hormones such as estrogen, testosterone, progesterone, glucocortisone, adrenaline, insulin, glucagon, cortisol, vitamin D, thyroid hormone, retinoic acid, and growth hormones; growth factors such as VEGF, EGF, NGF, and PDGF; cholesterol; bile acids; neurotransmitters such as GABA, Glutamate, acetylcholine; NOGO; inostitol triphosphate; diacylglycerol; epinephrine; norepinephrine; Nitric Oxide, peptides, vitamins such as folate and pyridoxine, drugs, antibodies and any other molecule that can interact with a receptor in vivo or in vitro. In one embodiment, the ligand can be attached to a compound of the invention using a linker molecule, such as an amide, amido, carbonyl, ester, peptide, disulphide, silane, nucleoside, abasic nucleoside, polyether, polyamine, polyamide, peptide, carbohydrate, lipid, polyhydrocarbon, phosphate ester, phosphoramidate, thiophosphate, alkylphosphate, or photolabile linker. In one embodiment, the linker is a biodegradable linker.

In one embodiment, the invention features a composition comprising a biologically active molecule complexed with a compound having any of Formula 1-60 or any combination thereof.

In one embodiment, the invention features a biologically active molecule complexed with a compound having any of Formula 1-60 or any combination thereof.

In one embodiment, a biologically active molecule of the invention comprises a siNA molecule or a portion thereof. In another embodiment, the siNA molecule is chemically modified (see for example Table II). In another embodiment, the siNA molecule does not comprise any ribonucleotides. Non-limiting examples of siNA molecules are described in McSwiggen, PCT/US04/16390, filed May 24, 2004 McSwiggen, U.S. Ser. No. 10/826,966, filed Apr. 16, 2004, McSwiggen et al., U.S. Ser. No. 10/444,853, filed May 23, 2003 and Vargeese et al., U.S. Ser. No. 10/427,160, filed Apr. 30, 2003, all of which are incorporated by reference herein in their entirety including the drawings.

In one embodiment, a biologically active molecule of the invention comprises an enzymatic nucleic acid.

In another embodiment, a biologically active molecule of the invention comprises an antisense nucleic acid, 2-5A nucleic acid chimera, decoy, aptamer, or a portion thereof.

In one embodiment, a composition of the invention comprises a compound or composition described in Beigelman et al., U.S. Pat. No. 6,395,713, and Beigelman et al., U.S. Ser. No. 10/036,916, both incorporated by reference herein in their entirety, including the drawings.

In one embodiment, the invention features a composition, comprising a biologically active molecule independently combined with one or more compounds having any of Formulae 1-60 in a suitable carrier or diluent. In another embodiment, the biologically active molecule is a nucleic acid, polynucleotide, oligonucleotide, peptide, polypeptide, protein, hormone, antibody, or small molecule. In another embodiment, the biologically active molecule is a siNA molecule or a portion thereof.

In one embodiment, the invention features a biologically active molecule, for example a siNA molecule, complexed with membrane disruptive agents such as those described in U.S. Patent Application Publication No. 20010007666, incorporated by reference herein in its entirety including the drawings. In another embodiment, the membrane disruptive agent or agents and the biologically active molecule are also complexed with a cationic lipid or helper lipid molecule, such as those lipids described in U.S. Pat. No. 6,235,310, incorporated by reference herein in its entirety including the drawings. In another embodiment, the membrane disruptive agent or agents and the biologically active molecule are also complexed with a compound having any of Formulae 1-60 herein.

In one embodiment, the invention features a method, comprising combining a biologically active molecule with one or more compounds having any of Formulae 1-60 under conditions suitable for the biologically active molecule to be complexed with the compound(s) having Formulae 1-60. In another embodiment, the biologically active molecule is a nucleic acid, polynucleotide, oligonucleotide, peptide, polypeptide, protein, hormone, antibody, or small molecule. In another embodiment, the biologically active molecule is a siNA molecule or a portion thereof.

In one embodiment, the invention features a method, comprising combining one or more compounds having any of Formulae 1-60 with a biologically active molecule under conditions suitable for the biologically active molecule to be complexed with the compound(s) having Formulae 1-60. In another embodiment, the biologically active molecule is a nucleic acid, polynucleotide, oligonucleotide, peptide, polypeptide, protein, hormone, antibody, or small molecule. In another embodiment, the biologically active molecule is a siNA molecule or a portion thereof. In one embodiment, one or more compounds having any of Formulae 1-60 is adjusted to a pH of about 7 before combining the biologically active molecule. In another embodiment, a molar excess (e.g. greater than two molar equivalents) of the compound(s) having any of Formulae 1-60 is combined with the biologically active molecule such that the biologically active molecule is completely ion paired with the compound(s) having any of Formulae 1-60. In another embodiment, a molar excess (e.g. greater than two molar equivalents) of the biologically active molecule is combined with the compound(s) having any of Formulae 1-60 such that the biologically active molecule is partially ion paired with the compound(s) having any of Formulae 1-60.

In one embodiment, the invention features a composition comprising a biologically active molecule complexed with a compound of the invention having any of Formulae 1-60 or any combination thereof and a pharmaceutically acceptable carrier or diluent.

In one embodiment, the invention features a lipoplex comprising a cationic component, a lipid component, and a biologically active molecule component (e.g. siNA). The cationic compounds of the invention (e.g. compounds having any of Formulae 1-60) can be formulated into a lipoplex comprising a cationic component, a lipid component, and a biologically active molecule component (e.g. siNA). The lipid component can comprise any amphipathic compound as is generally known in the art, or alternately lipid compounds described in U.S. Pat. No. 6,235,310 or U.S. Pat. No. 6,395,713. The formation of a lipoplex can lead to improved pharmacokinetic properties such as increased half life and increased serum stability of biologically active molecules to be delivered to relevant cells and tissues.

In another embodiment, the invention features a method of treating a subject, comprising contacting cells of the subject with a composition of the invention under conditions suitable for the treatment. This treatment can comprise the use of one or more other drug therapies under conditions suitable for the treatment. In one embodiment, the subject is treated for cancer. Cancer types contemplated by the instant invention include but are not limited to breast cancer, lung cancer, colorectal cancer, brain cancer, esophageal cancer, stomach cancer, bladder cancer, pancreatic cancer, cervical cancer, head and neck cancer, ovarian cancer, melanoma, lymphoma, glioma, or multidrug resistant cancers.

In one embodiment, the invention features a method of treating a subject infected with a virus, comprising contacting cells of the subject with a pharmaceutical composition of the invention, under conditions suitable for the treatment. This treatment can comprise the use of one or more other drug therapies under conditions suitable for the treatment. The viruses contemplated by the instant invention include but are not limited to HIV, HBV, HCV, CMV, RSV, HSV, poliovirus, influenza, rhinovirus, west nile virus, Ebola virus, foot and mouth virus, papilloma virus, and severe acute respiratory virus (SARS).

In one embodiment, the invention features a method of treating a subject having a disease or pathologic condition relating to gene expression (i.e. the over-expression or under-expression of a gene, or the expression of a mutant gene), comprising contacting cells of the subject with a pharmaceutical composition of the invention, under conditions suitable for the treatment. This treatment can comprise the use of one or more other drug therapies under conditions suitable for the treatment. The disease or pathologic condition can include cancer, infectious disease, autoimmunity, inflammation, endocrine disorders, muscular dystrophy, renal disease, pulmonary disease, cardiovascular disease, CNS injury, CNS disease, neurodegenerative disease such as Alzheimer's disease, Huntington disease, Parkinson's disease, ALS, and epilepsy; birth defects, aging, any other disease or condition related to gene expression.

The invention also features methods for generating compounds having Formulae 1-60. In one embodiment, the invention features methods for converting an amino group into a guanidinium group. Such methods can be used to generate compounds of the invention bearing guanidinium groups, such as a compound having any of Formulae 1-60, wherein X, Y, or Z is a guanidinium group.

In one embodiment, the invention features a method (guanidinium method 1) of introducing a guanidinium group to a compound comprising an amino group, such as a compound having any of Formulae 1-60 wherein any of X, Y, or Z is an amino group, comprising: (a) introducing a protecting group (P) at the primary amine of 1-H-pyrazole-1-carboxamidine or a salt thereof under conditions suitable to generate a protected 1-H-pyrazole-1-carboxamidine derivative; 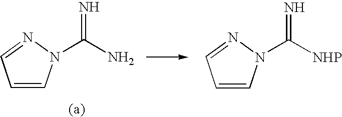
(b) coupling the product of (a) with a compound comprising a primary amine under conditions suitable for conversion of the amine to a protected guanidinium group; and 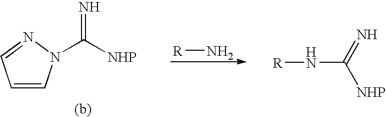
(c) deprotecting the guanidinium group under conditions suitable to isolate a compound comprising a guanidinum group or a salt thereof.

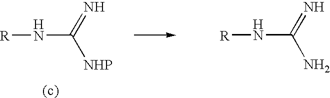

In one embodiment, the invention features a method (guanidinium method 2) of introducing a guanidinium group to a compound comprising an amino group, such as a compound having any of Formulae 1-60 wherein any of X, Y, or Z is an amino group, comprising: (a) introducing a protecting group (P) at the primary amine and the secondary amine of 1-H-pyrazole-1-carboxamidine or a salt thereof under conditions suitable to generate a bis-protected 1-H-pyrazole-1-carboxamidine derivative; 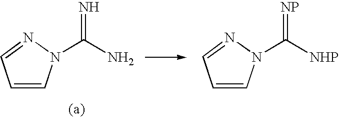
(b) coupling the product of (a) with a compound comprising a primary amine under conditions suitable for conversion of the amine to a bis-protected guanidinium group; and 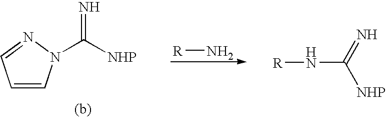
(c) deprotecting the guanidinium group under conditions suitable to isolate a compound comprising a guanidinum group or a salt thereof.

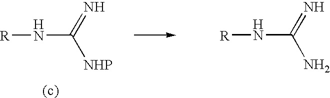

In one embodiment, the invention features a method (guanidinium method 3) of introducing a guanidinium group to a compound comprising an amino group, such as a compound having any of Formulae 1-60 wherein any of X, Y, or Z is an amino group, comprising: (a) introducing a protecting group (P) at the primary amine and the secondary amine of 1-H-pyrazole-1-carboxamidine or a salt thereof under conditions suitable to generate a bis-protected 1-H-pyrazole-1-carboxamidine derivative; 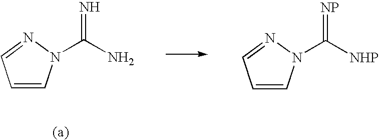
(b) coupling the product of (a) with a compound comprising a primary amine under conditions suitable for conversion of the amine to a bis-protected guanidinium group; and 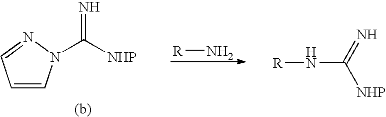
(c) selectively deprotecting one amine of the guanidinium group under conditions suitable to isolate a compound comprising a partially protected guanidinum group or a salt thereof.

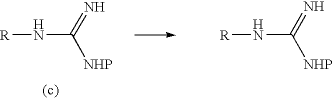

In one embodiment, R—NH2 shown in guanidinium methods 1-3 above comprises a compound having any of Formulae 1-60, wherein any of X, Y, or Z comprises a primary amine.

In one embodiment, R in guanidinium methods 1-3 above comprises a substituted or unsubstituted straight chain, branched chain, or cyclic alkyl, a polyether, a polyamine, or polyglycol having one or more primary amino groups, such as at either end of a linear compound or at differing ring positions of a cyclic compound (e.g. para, ortho, or meta substitution of a six membered ring).

In one embodiment, P shown in methods 1-3 above comprises an amino protecting group as is known in the art, such as a BOC, t-BOC, CBZ, or Fmoc protecting group.

In one embodiment, the invention features a method (guanidinium method 4) of introducing a guanidinium group to a compound comprising two amino groups, such as a compound having any of Formulae 1-19 or 38-60, wherein any of X, Y, or Z is an amino group, comprising: (a) introducing a protecting group (P) at the primary amine of 1-H-pyrazole-1-carboxamidine or a salt thereof under conditions suitable to generate a protected 1-H-pyrazole-1-carboxamidine derivative; 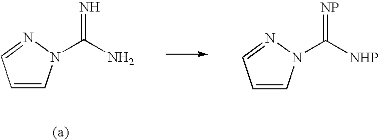
(b) coupling the product of (a) with a compound comprising two primary amine groups under conditions suitable for conversion of the amine groups to protected guanidinium groups; and 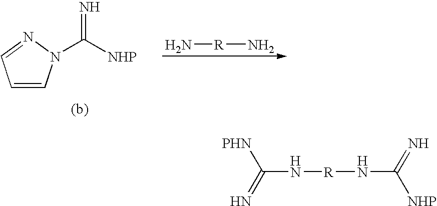
(c) deprotecting the guanidinium groups under conditions suitable to isolate a compound comprising two guanidinum groups or a salt thereof.

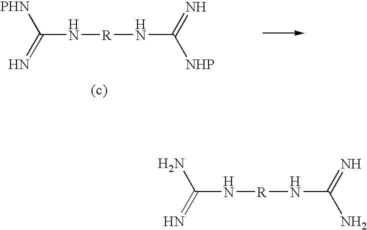

In one embodiment, the invention features a method (guanidinium method 5) of introducing a guanidinium group to a compound comprising two amino groups, such as a compound having any of Formulae 1-19 or 38-60, wherein any of X, Y, or Z is an amino group, comprising: (a) introducing a protecting group (P) at the primary amine and secondary amine of 1-H-pyrazole-1-carboxamidine or a salt thereof under conditions suitable to generate a bis-protected 1-H-pyrazole-1-carboxamidine derivative; 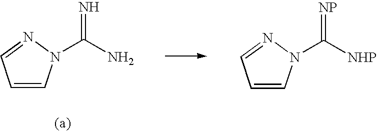
(b) coupling the product of (a) with a compound comprising two primary amine groups under conditions suitable for conversion of the amine groups to bis-protected guanidinium groups; and 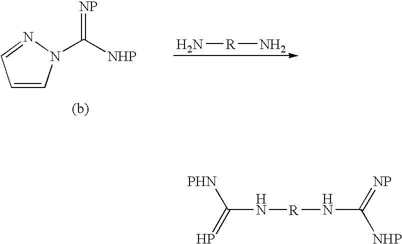
(c) deprotecting the guanidinium groups under conditions suitable to isolate a compound comprising two guanidinum groups or a salt thereof.

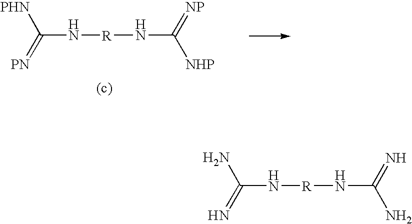

In one embodiment, the invention features a method (guanidinium method 6) of introducing a guanidinium group to a compound comprising two amino groups, such as a compound having any of Formulae 1-19 or 38-60, wherein any of X, Y, or Z is an amino group, comprising: (a) introducing a protecting group (P) at the primary amine and secondary amine of 1-H-pyrazole-1-carboxamidine or a salt thereof under conditions suitable to generate a bis-protected 1-H-pyrazole-1-carboxamidine derivative; 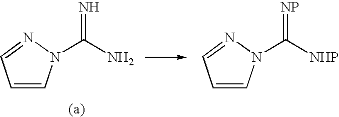
(b) coupling the product of (a) with a compound comprising two primary amine groups under conditions suitable for conversion of the amine groups to bis-protected guanidinium groups; and 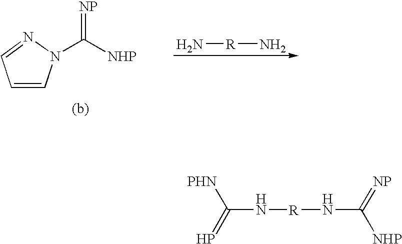
(c) selectively deprotecting one of the amines of the guanidinium groups under conditions suitable to isolate a compound comprising two partially protected guanidinum groups or a salt thereof.

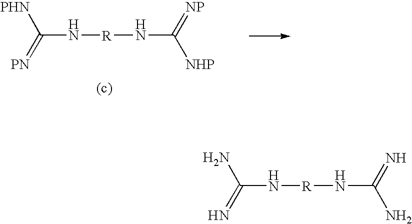

In one embodiment, H2N—R—NH2 shown in guanidinium methods 4-6 above comprises a compound having any of Formulae 1-19, 38 or 57, wherein both X and Y and/or Z comprise a primary amine.

In one embodiment, R in guanidinium methods 4-6 above comprises a substituted or unsubstituted straight chain, branched chain, or cyclic alkyl, a polyether, a polyamine, or polyglycol having one or more primary amino groups, such as at either end of a linear compound or at differing ring positions of a cyclic compound (e.g. para, ortho, or meta substitution of a six membered ring).

In one embodiment, P shown in guanidinium methods 4-6 above comprises an amino protecting group as is known in the art, such as a BOC, t-BOC, CBZ, or Fmoc protecting group.

The formulated compounds and compositions of the invention (e.g. complexes of compounds having Formulae 1-60 and a biologically active molecule) are added directly, or can be complexed with lipids, packaged within liposomes, or otherwise delivered to target cells or tissues. The compounds and compositions can be locally administered to relevant tissues ex vivo, or in vivo through injection or infusion pump, with or without their incorporation in biopolymers. The compounds and compositions of the instant invention, individually, or in combination or in conjunction with other drugs, can be used to treat diseases or conditions known in the art. For example, to treat a disease or condition associated with the levels of a protein or virus, the patient can be treated, or other appropriate cells can be treated, as is evident to those skilled in the art, individually or in combination with one or more drugs under conditions suitable for the treatment.

In a further embodiment, the described compositions and biologically active molecules of the invention can be used in combination with other known treatments to treat conditions or diseases. For example, the described molecules can be used in combination with one or more known therapeutic agents to treat breast, lung, prostate, colorectal, brain, esophageal, bladder, pancreatic, cervical, head and neck, and ovarian cancer, melanoma, lymphoma, glioma, multidrug resistant cancers, and/or HIV, HBV, HCV, CMV, RSV, HSV, poliovirus, influenza, rhinovirus, west nile virus, Ebola virus, foot and mouth virus, papilloma virus, and SARS virus infection, other cancers and other infectious diseases, autoimmunity, inflammation, endocrine disorders, renal disease, pulmonary disease, cardiovascular disease, CNS injury, CNS disease, neurodegenerative disease, birth defects, aging, any other disease or condition related to gene expression.

Included in another embodiment are a series of liposome formulations including one or more compounds of Formulae 1-60 herein that enhance the cellular uptake and transmembrane permeability of biologically active molecules in a variety of cell types. The formulated compounds and compositions of the invention (e.g. complexes of compounds having Formulae 1-60 and a biologically active molecule) are used either alone or in combination with other compounds with a neutral or a negative charge including but not limited to neutral lipid and/or targeting components, to improve the effectiveness of the formulation or composition in delivering and targeting the predetermined compound or molecule to cells. Another embodiment of the invention encompasses the utility of these compounds for increasing the transport of other impermeable and/or lipophilic compounds into cells. Targeting components include ligands for cell surface receptors including, peptides and proteins, glycolipids, lipids, steroid hormones, second messengers, carbohydrates, and their synthetic variants, for example growth factor, folate, cholesterol, signal peptide, or galactose receptors.

In another embodiment, the compounds (e.g. compounds having any of Formulae 1-60) of the invention are provided as a surface component of a lipid aggregate, covalently or ionically bound, such as a liposome encapsulated with the predetermined molecule to be delivered. Liposomes, which can be unilamellar or multilamellar, can introduce encapsulated material into a cell by different mechanisms. For example, the liposome can directly introduce its encapsulated material into the cell cytoplasm by fusing with the cell membrane. Alternatively, the liposome can be compartmentalized into an acidic vacuole (i.e., an endosome) and its contents released from the liposome and out of the acidic vacuole into the cellular cytoplasm.

In one embodiment the invention features a lipid aggregate formulation of the compounds (e.g. compounds having any of Formulae 1-60) and biologically active molecules described herein, including phosphatidylcholine (of varying chain length; e.g., egg yolk phosphatidylcholine), cholesterol, a cationic lipid, and 1,2-distearoyl-sn-glycero-3-phosphoethanolamine-polythyleneglycol-2000 (DSPE-PEG2000). The cationic lipid component of this lipid aggregate can be any cationic lipid known in the art such as dioleoyl 1,2,-diacyl-3-trimethylammonium-propane (DOTAP). In another embodiment this cationic lipid aggregate comprises a covalently bound compound described in any of the Formulae herein.

In another embodiment, polyethylene glycol (PEG) is covalently attached to the compounds (e.g. compounds having any of Formulae 1-60) of the present invention. The attached PEG can be any molecular weight but is preferably between 2000-50,000 daltons.

The compounds (e.g. compounds having any of Formulae 1-60) and methods of the present invention are useful for introducing nucleotides, nucleosides, nucleic acid molecules, polynucleotides, oligonucleotides, peptides, polypeptides, proteins, antibodies, lipids, and/or small molecule drugs into a cell. For example, the invention can be used for delivery of therapeutic compounds where the corresponding target site of action exists intracellularly.

In one embodiment, a compound of the invention is designed to be biodegradable, for example by introducing double bonds to saturated alkyl chains of compounds having any of Formulae 1-60 or by introducing chemical groups or linkers that are biodegradable.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of the invention also include molecules capable of modulating the pharmacokinetics and/or pharmacodynamics of other biologically active molecules, for example lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus containing group and saturated or unsaturated alkyl group, optionally substituted with OH, COOH, oxo, amine, or substituted or unsubstituted aryl groups.

The term “nitrogen containing group” as used herein refers to any chemical group or moiety comprising a nitrogen or substituted nitrogen. Non-limiting examples of nitrogen containing groups include amines, substituted amines, amides, alkylamines, amino acids such as arginine or lysine, polyamines such as spermine or spermidine, cyclic amines such as pyridines, pyrimidines including uracil, thymine, and cytosine, morpholines, phthalimides, and heterocyclic amines such as purines, including guanine and adenine.

The term “target molecule” as used herein, refers to nucleic acid molecules, proteins, peptides, antibodies, polysaccharides, lipids, sugars, metals, microbial or cellular metabolites, analytes, pharmaceuticals, and other organic and inorganic molecules that are present in a system.

By “inhibit” or “down-regulate” is meant that the expression of the gene, or level of RNAs or equivalent RNAs encoding one or more protein subunits, or activity of one or more protein subunits, such as pathogenic protein, viral protein or cancer related protein subunit(s), is reduced below that observed in the absence of the compounds or combination of compounds of the invention. In one embodiment, inhibition or down-regulation with an siNA molecule preferably is below that level observed in the presence of an inactive or scrambled siNA molecule. In another embodiment, inhibition or down-regulation with antisense oligonucleotides is preferably below that level observed in the presence of, for example, an oligonucleotide with scrambled sequence or with mismatches. In another embodiment, inhibition or down-regulation of viral or oncogenic RNA, protein, or protein subunits with a compound of the instant invention is greater in the presence of the compound than in its absence.

By “up-regulate” is meant that the expression of the gene, or level of RNAs or equivalent RNAs encoding one or more protein subunits, or activity of one or more protein subunits, such as viral or oncogenic protein subunit(s), is greater than that observed in the absence of the compounds or combination of compounds of the invention. For example, the expression of a gene, such as a viral or cancer related gene, can be increased in order to treat, prevent, ameliorate, or modulate a pathological condition caused or exacerbated by an absence or low level of gene expression.

By “modulate” is meant that the expression of the gene, or level of RNAs or equivalent RNAs encoding one or more protein subunits, or activity of one or more protein subunit(s) of a protein, for example a viral or cancer related protein is up-regulated or down-regulated, such that the expression, level, or activity is greater than or less than that observed in the absence of the compounds or combination of compounds of the invention.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of inhibiting or down regulating gene expression or viral replication, for example by mediating RNA interference “RNAi” or gene silencing in a sequence-specific manner; see for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). For example the siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 15 to about 30, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the siNA molecule are complementary to the target nucleic acid or a portion thereof). Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the siNA molecules of the invention comprise nucleotide sequence that is complementary to nucleotide sequence of a target gene. In another embodiment, the siNA molecule of the invention interacts with nucleotide sequence of a target gene in a manner that causes inhibition of expression of the target gene. As used herein, siNA molecules need not be limited to those molecules containing only RNA, but further encompasses chemically-modified nucleotides and non-nucleotides. In certain embodiments, the short interfering nucleic acid molecules of the invention lack 2′-hydroxy (2′-OH) containing nucleotides. Applicant describes in certain embodiments short interfering nucleic acids that do not require the presence of nucleotides having a 2′-hydroxy group for mediating RNAi and as such, short interfering nucleic acid molecules of the invention optionally do not include any ribonucleotides (e.g., nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions. The modified short interfering nucleic acid molecules of the invention can also be referred to as short interfering modified oligonucleotides “siMON.” As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, siNA molecules of the invention can be used to epigenetically silence genes at both the post-transcriptional level or the pre-transcriptional level. In a non-limiting example, epigenetic regulation of gene expression by siNA molecules of the invention can result from siNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

In one embodiment, a siNA molecule of the invention is a duplex forming oligonucleotide “DFO”, (see, for example, Vaish et al., U.S. Ser. No. 10/727,780 filed Dec. 3, 2003 and International PCT Application No. US04/16390, filed May 24, 2004, both of which are hereby incorporated by reference herein in their entireties, including the drawings).

In one embodiment, a siNA molecule of the invention is a multifunctional siNA, (see for example Jadhav et al., U.S. Ser. No. 60/543,480 filed Feb. 10, 2004 and International PCT Application No. US04/16390, filed May 24, 2004, both of which are hereby incorporated by reference herein in their entireties, including the drawings). The multifunctional siNA of the invention can comprise sequence targeting, for example, two regions of RNA.

The term “enzymatic nucleic acid molecule” as used herein refers to a nucleic acid molecule which has complementarity in a substrate binding region to a specified gene target, and also has an enzymatic activity which is active to specifically cleave target RNA. That is, the enzymatic nucleic acid molecule is able to intermolecularly cleave RNA and thereby inactivate a target RNA molecule. These complementary regions allow sufficient hybridization of the enzymatic nucleic acid molecule to the target RNA and thus permit cleavage. One hundred percent complementarity is preferred, but complementarity as low as 50-75% can also be useful in this invention (see for example Werner and Uhlenbeck, 1995, Nucleic Acids Research, 23, 2092-2096; Hammann et al., 1999, Antisense and Nucleic Acid Drug Dev., 9, 25-31). The nucleic acids can be modified at the base, sugar, and/or phosphate groups. The term enzymatic nucleic acid is used interchangeably with phrases such as ribozymes, catalytic RNA, enzymatic RNA, catalytic DNA, aptazyme or aptamer-binding ribozyme, regulatable ribozyme, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving and/or ligation activity to the molecule (Cech et al., U.S. Pat. No. 4,987,071; Cech et al., 1988, 260 JAMA 3030).

By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complimentary nucleotides to base pair with the antisense region and form a duplex with loop. For example, an asymmetric hairpin siNA molecule of the invention can comprise an antisense region having length sufficient to mediate RNAi in a cell or in vitro system (e.g. about 19 to about 22 nucleotides) and a loop region comprising about 4 to about 8 nucleotides, and a sense region having about 3 to about 18 nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complimentary nucleotides to base pair with the antisense region and form a duplex. For example, an asymmetric duplex siNA molecule of the invention can comprise an antisense region having length sufficient to mediate RNAi in a cell or in vitro system (e.g. about 19 to about 22 nucleotides) and a sense region having about 3 to about 18 nucleotides that are complementary to the antisense region.

The term “nucleic acid molecule” as used herein, refers to a molecule having nucleotides. The nucleic acid can be single, double, or multiple stranded and can comprise modified or unmodified nucleotides or non-nucleotides or various mixtures and combinations thereof.

The term “enzymatic portion” or “catalytic domain” as used herein refers to that portion/region of the enzymatic nucleic acid molecule essential for cleavage of a nucleic acid substrate.

The term “substrate binding arm” or “substrate binding domain” as used herein refers to that portion/region of a enzymatic nucleic acid which is able to interact, for example via complementarity (i.e., able to base-pair with), with a portion of its substrate. Preferably, such complementarity is 100%, but can be less if desired. For example, as few as 10 bases out of 14 can be base-paired (see for example Werner and Uhlenbeck, 1995, Nucleic Acids Research, 23, 2092-2096; Hammann et al., 1999, Antisense and Nucleic Acid Drug Dev., 9, 25-31). Examples of such arms are shown generally in FIGS. 1-4. That is, these arms contain sequences within a enzymatic nucleic acid which are intended to bring enzymatic nucleic acid and target RNA together through complementary base-pairing interactions. The enzymatic nucleic acid of the invention can have binding arms that are contiguous or non-contiguous and can be of varying lengths. The length of the binding arm(s) are preferably greater than or equal to four nucleotides and of sufficient length to stably interact with the target RNA; preferably 12-100 nucleotides; more preferably 14-24 nucleotides long (see for example Werner and Uhlenbeck, supra; Hamman et al., supra; Hampel et al., EP0360257; Berzal-Herrance et al., 1993, EMBO J., 12, 2567-73). If two binding arms are chosen, the design is such that the length of the binding arms are symmetrical (i.e., each of the binding arms is of the same length; e.g., five and five nucleotides, or six and six nucleotides, or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

The term “sufficient length” as used herein, refers to an oligonucleotide of length great enough to provide the intended function under the expected condition, i.e., greater than or equal to 3 nucleotides. For example, for binding arms of enzymatic nucleic acid “sufficient length” means that the binding arm sequence is long enough to provide stable binding to a target site under the expected binding conditions. Preferably, the binding arms are not so long as to prevent useful turnover of the nucleic acid molecule. In another example, the length of a siNA molecule is of length sufficient to mediate RNAi activity.

The term “stably interact” as used herein, refers to interaction of the oligonucleotides with target nucleic acid (e.g., by forming hydrogen bonds with complementary nucleotides in the target under physiological conditions) that is sufficient to the intended purpose (e.g., mediated of RNAi or cleavage of target RNA by an enzyme).

The term “homology” as used herein, refers to the nucleotide sequence of two or more nucleic acid molecules is partially or completely identical.

The term “antisense nucleic acid”, as used herein, refers to a non-enzymatic nucleic acid molecule that binds to target RNA by means of RNA-RNA or RNA-DNA or RNA-PNA (protein nucleic acid; Egholm et al., 1993 Nature 365, 566) interactions and alters the activity of the target RNA (for a review, see Stein and Cheng, 1993 Science 261, 1004 and Woolf et al., U.S. Pat. No. 5,849,902). Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. For a review of current antisense strategies, see Schmajuk et al., 1999, J. Biol. Chem., 274, 21783-21789, Delihas et al., 1997, Nature, 15, 751-753, Stein et al., 1997, Antisense N. A. Drug Dev., 7, 151, Crooke, 2000, Methods Enzymol., 313, 3-45; Crooke, 1998, Biotech. Genet. Eng. Rev., 15, 121-157, Crooke, 1997, Ad. Pharmacol., 40, 1-49. In addition, antisense DNA can be used to target RNA by means of DNA-RNA interactions, thereby activating RNase H, which digests the target RNA in the duplex. The antisense oligonucleotides can comprise one or more RNAse H activating region, which is capable of activating RNAse H cleavage of a target RNA. Antisense DNA can be synthesized chemically or expressed via the use of a single stranded DNA expression vector or equivalent thereof.

The term “RNase H activating region” as used herein, refers to a region (generally greater than or equal to 4-25 nucleotides in length, preferably from 5-11 nucleotides in length) of a nucleic acid molecule capable of binding to a target RNA to form a non-covalent complex that is recognized by cellular RNase H enzyme (see for example Arrow et al., U.S. Pat. No. 5,849,902; Arrow et al., U.S. Pat. No. 5,989,912). The RNase H enzyme binds to the nucleic acid molecule-target RNA complex and cleaves the target RNA sequence. The RNase H activating region comprises, for example, phosphodiester, phosphorothioate (preferably at least four of the nucleotides are phosphorothiote substitutions; more specifically, 4-11 of the nucleotides are phosphorothiote substitutions); phosphorodithioate, 5′-thiophosphate, or methylphosphonate backbone chemistry or a combination thereof. In addition to one or more backbone chemistries described above, the RNase H activating region can also comprise a variety of sugar chemistries. For example, the RNase H activating region can comprise deoxyribose, arabino, fluoroarabino or a combination thereof, nucleotide sugar chemistry. Those skilled in the art will recognize that the foregoing are non-limiting examples and that any combination of phosphate, sugar and base chemistry of a nucleic acid that supports the activity of RNase H enzyme is within the scope of the definition of the RNase H activating region and the instant invention.

The term “2-5A antisense chimera” as used herein, refers to an antisense oligonucleotide containing a 5′-phosphorylated 2′-5′-linked adenylate residue. These chimeras bind to target RNA in a sequence-specific manner and activate a cellular 2-5A-dependent ribonuclease which, in turn, cleaves the target RNA (Torrence et al., 1993 Proc. Natl. Acad. Sci. USA 90, 1300; Silverman et al., 2000, Methods Enzymol., 313, 522-533; Player and Torrence, 1998, Pharmacol. Ther., 78, 55-113).

The term “triplex forming oligonucleotides” as used herein, refers to an oligonucleotide that can bind to a double-stranded DNA in a sequence-specific manner to form a triple-strand helix. Formation of such triple helix structure has been shown to inhibit transcription of the targeted gene (Duval-Valentin et al., 1992 Proc. Natl. Acad. Sci. USA 89, 504; Fox, 2000, Curr. Med. Chem., 7, 17-37; Praseuth et. al., 2000, Biochim. Biophys. Acta, 1489, 181-206).

The term “gene” it as used herein, refers to a nucleic acid that encodes an RNA, for example, nucleic acid sequences including but not limited to structural genes encoding a polypeptide.

The term “pathogenic protein” as used herein, refers to endogenous or exogenous proteins that are associated with a disease state or condition, for example a particular cancer or viral infection.

The term “complementarity” refers to the ability of a nucleic acid to form hydrogen bond(s) with another RNA sequence by either traditional Watson-Crick or other non-traditional types. In reference to the nucleic molecules of the present invention, the binding free energy for a nucleic acid molecule with its target or complementary sequence is sufficient to allow the relevant function of the nucleic acid to proceed, e.g., RNA interference, enzymatic nucleic acid cleavage, antisense or triple helix inhibition. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence.

By “RNA” is meant a molecule comprising at least one ribonucleotide residue. By “ribonucleotide” is meant a nucleotide with a hydroxyl group at the 2′ position of a β-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “ribonucleotide” or “2′-OH” is meant a nucleotide with a hydroxyl group at the 2′ position of a β-D-ribo-furanose moiety.

The term “decoy RNA” as used herein, refers to a RNA molecule or aptamer that is designed to preferentially bind to a predetermined ligand. Such binding can result in the inhibition or activation of a target molecule. The decoy RNA or aptamer can compete with a naturally occurring binding target for the binding of a specific ligand. For example, it has been shown that over-expression of HIV trans-activation response (TAR) RNA can act as a “decoy” and efficiently binds HIV tat protein, thereby preventing it from binding to TAR sequences encoded in the HIV RNA (Sullenger et al., 1990, Cell, 63, 601-608). This is but a specific example and those in the art will recognize that other embodiments can be readily generated using techniques generally known in the art, see for example Gold et al., 1995, Annu. Rev. Biochem., 64, 763; Brody and Gold, 2000, J. Biotechnol., 74, 5; Sun, 2000, Curr. Opin. Mol. Ther., 2, 100; Kusser, 2000, J. Biotechnol., 74, 27; Hermann and Patel, 2000, Science, 287, 820; and Jayasena, 1999, Clinical Chemistry, 45, 1628. Similarly, a decoy RNA can be designed to bind to a receptor and block the binding of an effector molecule or a decoy RNA can be designed to bind to receptor of interest and prevent interaction with the receptor.

The term “single stranded RNA” (ssRNA) as used herein refers to a naturally occurring or synthetic ribonucleic acid molecule comprising a linear single strand, for example a ssRNA can be a messenger RNA (mRNA), transfer RNA (tRNA), ribosomal RNA (rRNA) etc. of a gene.

The term “single stranded DNA” (ssDNA) as used herein refers to a naturally occurring or synthetic deoxyribonucleic acid molecule comprising a linear single strand, for example, a ssDNA can be a sense or antisense gene sequence or EST (Expressed Sequence Tag).

The term “double stranded RNA” or “dsRNA” as used herein refers to a double stranded RNA molecule capable of RNA interference, including short interfering RNA (siNA).

The term “allozyme” as used herein refers to an allosteric enzymatic nucleic acid molecule, see for example see for example George et al., U.S. Pat. Nos. 5,834,186 and 5,741,679, Shih et al., U.S. Pat. No. 5,589,332, Nathan et al., U.S. Pat. No. 5,871,914, Nathan and Ellington, International PCT publication No. WO 00/24931, Breaker et al., International PCT Publication Nos. WO 00/26226 and 98/27104, and Sullenger et al., International PCT publication No. WO 99/29842.

The term “cell” as used herein, refers to its usual biological sense, and does not refer to an entire multicellular organism. The cell can, for example, be in vitro, e.g., in cell culture, or present in a multicellular organism, including, e.g., birds, plants and mammals such as humans, cows, sheep, apes, monkeys, swine, dogs, and cats. The cell can be prokaryotic (e.g., bacterial cell) or eukaryotic (e.g., mammalian or plant cell).

The term “highly conserved sequence region” as used herein, refers to a nucleotide sequence of one or more regions in a target gene does not vary significantly from one generation to the other or from one biological system to the other.

The term “non-nucleotide” as used herein, refers to any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine.

The term “nucleotide” as used herein, refers to a heterocyclic nitrogenous base in N-glycosidic linkage with a phosphorylated sugar. Nucleotides are recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, for example, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

The term “nucleoside” as used herein, refers to a heterocyclic nitrogenous base in N-glycosidic linkage with a sugar. Nucleosides are recognized in the art to include natural bases (standard), and modified bases well known in the art. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, modified nucleosides, non-natural nucleosides, non-standard nucleosides and other; see for example, Usman and McSwiggen, supra; Eckstein et al., International PCT Publication No. WO 92/07065; Usman et al., International PCT Publication No. WO 93/15187; Uhlman & Peyman, supra all are hereby incorporated by reference herein). There are several examples of modified nucleic acid bases known in the art as summarized by Limbach et al., 1994, Nucleic Acids Res. 22, 2183. Some of the non-limiting examples of chemically modified and other natural nucleic acid bases that can be introduced into nucleic acids include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2methylthio-N-6-isopentenyladenosine, beta-D-mannosylqueosine, uridine-5-oxyacetic acid, 2-thiocytidine, threonine derivatives and others (Burgin et al., 1996, Biochemistry, 35, 14090; Uhlman & Peyman, supra). By “modified bases” in this aspect is meant nucleoside bases other than adenine, guanine, cytosine and uracil at 1′ position or their equivalents; such bases can be used at any position, for example, within the catalytic core of an enzymatic nucleic acid molecule and/or in the substrate-binding regions of the nucleic acid molecule.

The term “cap structure” as used herein, refers to chemical modifications, which have been incorporated at either terminus of the oligonucleotide (see for example Wincott et al., WO 97/26270, incorporated by reference herein). These terminal modifications protect the nucleic acid molecule from exonuclease degradation, and can help in delivery and/or localization within a cell. The cap can be present at the 5′-terminus (5′-cap) or at the 3′-terminus (3′-cap) or can be present on both terminus. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270, incorporated by reference herein).

The term “abasic” as used herein, refers to sugar moieties lacking a base or having other chemical groups in place of a base at the 1′ position, for example a 3′,3′-linked or 5′,5′-linked deoxyabasic ribose derivative (for more details see Wincott et al., International PCT publication No. WO 97/26270).

The term “unmodified nucleoside” as used herein, refers to one of the bases adenine, cytosine, guanine, thymine, uracil joined to the 1′ carbon of β-D-ribo-furanose.

The term “modified nucleoside” as used herein, refers to any nucleotide base which contains a modification in the chemical structure of an unmodified nucleotide base, sugar and/or phosphate.

By “subject” is meant an organism, which is a donor or recipient of explanted cells or the cells themselves. “Subject” also refers to an organism to which the nucleic acid molecules of the invention can be administered. A subject can be a mammal or mammalian cells, including a human or human cells.

The term “enhanced enzymatic activity” as used herein, includes activity measured in cells and/or in vivo where the activity is a reflection of both the catalytic activity and the stability of the nucleic acid molecules of the invention. In this invention, the product of these properties can be increased in vivo compared to an all RNA enzymatic nucleic acid or all DNA enzyme. In some cases, the activity or stability of the nucleic acid molecule can be decreased (i.e., less than ten-fold), but the overall activity of the nucleic acid molecule is enhanced, in vivo.

The term “negatively charged molecules” as used herein, refers to molecules such as nucleic acid molecules (e.g., RNA, DNA, oligonucleotides, mixed polymers, peptide nucleic acid, and the like), peptides (e.g., polyaminoacids, polypeptides, proteins and the like), nucleotides, pharmaceutical and biological compositions, that have negatively charged groups that can ion-pair with the positively charged head group of the cationic lipids of the invention.

The term “coupling” as used herein, refers to a reaction, either chemical or enzymatic, in which one atom, moiety, group, compound or molecule is joined to another atom, moiety, group, compound or molecule.

The terms “deprotection” or “deprotecting” as used herein, refers to the removal of a protecting group.

The term “guanidinium” refers to a chemical group having the general formula: 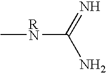
including any salts thereof and where R is H, or wherein the term “substituted guandinium” is employed, R is alkyl or substituted alkyl.

The term “histidyl” refers to a chemical group having the general formula: 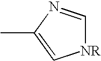
including any salts thereof and where R is H, or wherein the term “substituted histidyl” is employed, R is alkyl or substituted alkyl.

The term “alkyl” as used herein refers to a saturated aliphatic hydrocarbon, including straight-chain, branched-chain “isoalkyl”, and cyclic alkyl groups. The term “alkyl” also comprises alkoxy, alkyl-thio, alkyl-thio-alkyl, alkoxyalkyl, alkylamino, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. In one embodiment, the alkyl group can comprise 1 to 12 carbons. In another embodiment, the alkyl is a lower alkyl of from about 1 to about 7 carbons, or about 1 to about 4 carbons. The alkyl group can be substituted or unsubstituted. When substituted, the substituted group(s) can comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkenyl groups containing at least one carbon-carbon double bond, including straight-chain, branched-chain, and cyclic groups. In one embodiment, the alkenyl group has about 2 to about 12 carbons. In another embodiment, the alkenyl is a lower alkenyl of from about 2 to about 7 carbons, or about 2 to about 4 carbons. The alkenyl group can be substituted or unsubstituted. When substituted, the substituted group(s) can comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. The term “alkyl” also includes alkynyl groups containing at least one carbon-carbon triple bond, including straight-chain, branched-chain, and cyclic groups. In one embodiment, the alkynyl group has about 2 to about 12 carbons. In another embodiment, the alkynyl is a lower alkynyl of from about 2 to about 7 carbons, or about 2 to about 4 carbons. The alkynyl group can be substituted or unsubstituted. When substituted the substituted group(s) can comprise hydroxy, oxy, thio, amino, nitro, cyano, alkoxy, alkyl-thio, alkyl-thio-alkyl, alkoxyalkyl, alkylamino, silyl, alkenyl, alkynyl, alkoxy, cycloalkenyl, cycloalkyl, cycloalkylalkyl, heterocycloalkyl, heteroaryl, C1-C6 hydrocarbyl, aryl or substituted aryl groups. Alkyl groups or moieties of the invention can also include aryl, alkylaryl, carbocyclic aryl, heterocyclic aryl, amide and ester groups. The substituent(s) of aryl groups can comprise halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from about 1 to about 3 heteroatoms as ring atoms in the aromatic ring and the remainder of the ring atoms are carbon atoms. Suitable heteroatoms include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

The term “alkoxyalkyl” as used herein refers to an alkyl-O-alkyl ether, for example, methoxyethyl or ethoxymethyl.

The term “alkyl-thio-alkyl” as used herein refers to an alkyl-5-alkyl thioether, for example, methylthiomethyl or methylthioethyl.

The term “amino” as used herein refers to a nitrogen containing group as is known in the art derived from ammonia by the replacement of one or more hydrogen radicals by organic radicals. For example, the terms “aminoacyl” and “aminoalkyl” refer to specific N-substituted organic radicals with acyl and alkyl substituent groups respectively. By “amine” is meant a radical with the general formula —NHR or —NR₂, wherein each R is independently hydrogen, or wherein the term “substituted amine” is employed, R is alkyl or substituted alkyl.

The term “amination” as used herein refers to a process in which an amino group or substituted amine is introduced into an organic molecule.

The term “complex” refers to a mixture of one or more compounds that are associated via covalent or non-covalent interactions such as electrostatic interactions, hydrogen bonding interactions, and hydrophobic interactions. The term “complexed” refers to the process of combining one or more compounds to generate a complex. A complexed formulation or composition of the invention can be designed to have a net positive charge, a net negative charge, or a neutral charge depending on the ratio of differing compounds used to generate the complex. For example, a compound having any for Formulae 1-60 can be combined with a biologically active molecule and optionally another molecule, such as a lipid, to form a complex in a form or manner suitable for administration to a cell or subject.

The term “exocyclic amine protecting moiety” as used herein refers to a nucleobase amino protecting group compatible with oligonucleotide synthesis, for example, an acyl or amide group.

The term “alkenyl” as used herein refers to a straight or branched hydrocarbon of a designed number of carbon atoms containing at least one carbon-carbon double bond. Examples of “alkenyl” include vinyl, allyl, and 2-methyl-3-heptene.

The term “alkoxy” as used herein refers to an alkyl group of indicated number of carbon atoms attached to the parent molecular moiety through an oxygen bridge. Examples of alkoxy groups include, for example, methoxy, ethoxy, propoxy and isopropoxy.

The term “alkynyl” as used herein refers to a straight or branched hydrocarbon of a designed number of carbon atoms containing at least one carbon-carbon triple bond. Examples of “alkynyl” include propargyl, propyne, and 3-hexyne.

The term “aryl” as used herein refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

The term “cycloalkenyl” as used herein refers to a C3-C8 cyclic hydrocarbon containing at least one carbon-carbon double bond. Examples of cycloalkenyl include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “cycloalkyl” as used herein refers to a C3-C8 cyclic hydrocarbon. Examples of cycloalkyl include cyclopropyl, cyclobutyl, cyclopentyl, cyclohexyl, cycloheptyl and cyclooctyl.

The term “cycloalkylalkyl,” as used herein, refers to a C3-C7 cycloalkyl group attached to the parent molecular moiety through an alkyl group, as defined above. Examples of cycloalkylalkyl groups include cyclopropylmethyl and cyclopentylethyl.

The terms “halogen” or “halo” as used herein refers to indicate fluorine, chlorine, bromine, and iodine.

The term “heterocycloalkyl,” as used herein refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrolidinyl.

The term “heteroaryl” as used herein refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

The term “C1-C6 hydrocarbyl” as used herein refers to straight, branched, or cyclic alkyl groups having 1-6 carbon atoms, optionally containing one or more carbon-carbon double or triple bonds. Examples of hydrocarbyl groups include, for example, methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, vinyl, 2-pentene, cyclopropylmethyl, cyclopropyl, cyclohexylmethyl, cyclohexyl and propargyl. When reference is made herein to C₁-C₆ hydrocarbyl containing one or two double or triple bonds it is understood that at least two carbons are present in the alkyl for one double or triple bond, and at least four carbons for two double or triple bonds.

The term “protecting group” as used herein, refers to groups known in the art that are readily introduced and removed from an atom, for example O, N, P, or S. Protecting groups are used to prevent undesirable reactions from taking place that can compete with the formation of a specific compound or intermediate of interest. See also “Protective Groups in Organic Synthesis”, 3rd Ed., 1999, Greene, T. W. and related publications.

The term “nitrogen protecting group,” as used herein, refers to groups known in the art that are readily introduced on to and removed from a nitrogen. Examples of nitrogen protecting groups include Boc, Cbz, benzoyl, and benzyl. See also “Protective Groups in Organic Synthesis”, 3rd Ed., 1999, Greene, T. W. and related publications.

The term “hydroxy protecting group,” or “hydroxy protection” as used herein, refers to groups known in the art that are readily introduced on to and removed from an oxygen, specifically an —OH group. Examples of hyroxy protecting groups include trityl or substituted trityl goups, such as monomethoxytrityl and dimethoxytrityl, or substituted silyl groups, such as tert-butyldimethyl, trimethylsilyl, or tert-butyldiphenyl silyl groups. See also “Protective Groups in Organic Synthesis”, 3rd Ed., 1999, Greene, T. W. and related publications.

The term “acyl” as used herein refers to —C(O)R groups, wherein R is an alkyl or aryl.

The term “phosphorus containing group” as used herein, refers to a chemical group containing a phosphorus atom. The phosphorus atom can be trivalent or pentavalent, and can be substituted with O, H, N, S, C or halogen atoms. Examples of phosphorus containing groups of the instant invention include but are not limited to phosphorus atoms substituted with O, H, N, S, C or halogen atoms, comprising phosphonate, alkylphosphonate, phosphate, diphosphate, triphosphate, pyrophosphate, phosphorothioate, phosphorodithioate, phosphoramidate, phosphoramidite groups, nucleotides and nucleic acid molecules.

The term “ligand” refers to any compound or molecule, such as a drug, peptide, hormone, or neurotransmitter, that is capable of interacting with another compound, such as a receptor, either directly or indirectly. The receptor that interacts with a ligand can be present on the surface of a cell or can alternately be an intercellular receptor. Interaction of the ligand with the receptor can result in a biochemical reaction, or can simply be a physical interaction or association. Non-limiting examples of ligands include sugars and carbohydrates such as galactose, galactosamine, and N-acetyl galactosamine; hormones such as estrogen, testosterone, progesterone, glucocortisone, adrenaline, insulin, glucagon, cortisol, vitamin D, thyroid hormone, retinoic acid, and growth hormones; growth factors such as VEGF, EGF, NGF, and PDGF; cholesterol; bile acids; neurotransmitters such as GABA, Glutamate, acetylcholine; NOGO; inostitol triphosphate; diacylglycerol; epinephrine; norepinephrine; Nitric Oxide, peptides, vitamins such as folate and pyridoxine, drugs, antibodies and any other molecule that can interact with a receptor in vivo or in vitro. The ligand can be attached to a compound of the invention using a linker molecule, such as an amide, amido, carbonyl, ester, peptide, disulphide, silane, nucleoside, abasic nucleoside, polyether, polyamine, polyamide, peptide, carbohydrate, lipid, polyhydrocarbon, phosphate ester, phosphoramidate, thiophosphate, alkylphosphate, or photolabile linker. In one embodiment, the linker is a biodegradable linker.

The term “cationic salt” as used herein refers to any organic or inorganic salt having a net positive charge, for example a triethylammonium (TEA) salt.

The term “degradable linker” as used herein, refers to linker moieties that are capable of cleavage under various conditions. Conditions suitable for cleavage can include but are not limited to pH, UV irradiation, enzymatic activity, temperature, hydrolysis, elimination, and substitution reactions, and thermodynamic properties of the linkage.

The term “photolabile linker” as used herein, refers to linker moieties as are known in the art, that are selectively cleaved under particular UV wavelengths. Compounds of the invention containing photolabile linkers can be used to deliver compounds to a target cell or tissue of interest, and can be subsequently released in the presence of a UV source.

The term “lipid” as used herein, refers to any lipophilic compound. Non-limiting examples of lipid compounds include fatty acids and their derivatives, including straight chain, branched chain, saturated and unsaturated fatty acids, carotenoids, terpenes, bile acids, and steroids, including cholesterol and derivatives or analogs thereof.

The term “folate” as used herein, refers to analogs and derivatives of folic acid, for example antifolates, dihydrofloates, tetrahydrofolates, tetrahydorpterins, folinic acid, pteropolyglutamic acid, 1-deza, 3-deaza, 5-deaza, 8-deaza, 10-deaza, 1,5-deaza, 5,10 dideaza, 8,10-dideaza, and 5,8-dideaza folates, antifolates, and pteroic acid derivatives.

The term “compounds with neutral charge” as used herein, refers to compositions which are neutral or uncharged at neutral or physiological pH. Examples of such compounds are cholesterol and other steroids, cholesteryl hemisuccinate (CHEMS), dioleoyl phosphatidyl choline, distearoylphosphotidyl choline (DSPC), fatty acids such as oleic acid, phosphatidic acid and its derivatives, phosphatidyl serine, polyethylene glycol-conjugated phosphatidylamine, phosphatidylcholine, phosphatidylethanolamine and related variants, prenylated compounds including farnesol, polyprenols, tocopherol, and their modified forms, diacylsuccinyl glycerols, fusogenic or pore forming peptides, dioleoylphosphotidylethanolamine (DOPE), ceramide and the like.

The term “lipid aggregate” as used herein refers to a lipid-containing composition wherein the lipid is in the form of a liposome, micelle (non-lamellar phase) or other aggregates with one or more lipids.

The term “biological system” as used herein, refers to a eukaryotic system or a prokaryotic system, can be a bacterial cell, plant cell or a mammalian cell, or can be of plant origin, mammalian origin, yeast origin, Drosophila origin, or archebacterial origin.

The term “systemic administration” as used herein refers to the in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages are also useful. This approach can provide enhanced delivery of the drug to target cells by taking advantage of the specificity of macrophage and lymphocyte immune recognition of abnormal cells, such as the cancer cells.

The term “pharmacological composition” or “pharmaceutical formulation” refers to a composition or formulation in a form suitable for administration, for example, systemic administration, into a cell or patient, preferably a human. Suitable forms, in part, depend upon the use or the route of entry, for example oral, transdermal, or by injection. Such forms should not prevent the composition or formulation to reach a target cell (i.e., a cell to which the negatively charged polymer is targeted).

Other features and advantages of the invention will be apparent from the following description of the preferred embodiments thereof, and from the claims.

### BRIEF DESCRIPTION OF THE DRAWINGS

FIG. 1 shows non-limiting examples of cationic delivery reagents of the invention. Diamine (1) can be converted to a bis-guanidinium derivative (2), or alternately a guanidinium derivative (3), that can be further conjugated with a ligand (L) to generate (4). Any of compounds 1-4 can be complexed with a compound of interest, such as a nucleic acid or siNA construct, to generate a composition to facilitate the cellular delivery of the compound of interest. Other diamines having differing alkyl chain lengths can be similarly used to generate a variety of diamino, bis-guanidinium, guanidinium, and ligand derivatized complexes.

FIG. 2 shows non-limiting examples of cationic delivery reagents of the invention. Diamine (5) can be converted to a bis-guanidinium derivative (6), or alternately a guanidinium derivative (7), that can be further conjugated with a ligand (L) to generate (8). Any of compounds 5-8 can be complexed with a compound of interest, such as a nucleic acid or siNA construct, to generate a composition to facilitate the cellular delivery of the compound of interest. Other diamines having differing alkyl chain lengths or glycol composition can be similarly used to generate a variety of diamino, bis-guanidinium, guanidinium, and ligand derivatized complexes.

FIG. 3 shows non-limiting examples of cationic delivery reagents of the invention. Diamine (9) can be converted to a bis-guanidinium derivative (10), or alternately a guanidinium derivative (11), that can be further conjugated with a ligand (L) to generate (12). Any of compounds 9-12 can be complexed with a compound of interest, such as a nucleic acid or siNA construct, to generate a composition to facilitate the cellular delivery of the compound of interest. Other diamine ethers having differing alkyl chain lengths can be similarly used to generate a variety of diamino, bis-guanidinium, guanidinium, and ligand derivatized complexes.

FIG. 4 shows non-limiting examples of cationic delivery reagents of the invention. Polyamine (13) can be converted to a bis-guanidinium derivative (14), or alternately a guanidinium derivative (15), that can be further conjugated with a ligand (L) to generate (16). Any of compounds 13-16 can be complexed with a compound of interest, such as a nucleic acid or siNA construct, to generate a composition to facilitate the cellular delivery of the compound of interest. Other polyamines having differing alkyl chain lengths and nitrogen content can be similarly used to generate a variety of diamino, bis-guanidinium, guanidinium, and ligand derivatized complexes.

FIG. 5 shows non-limiting examples of cationic delivery reagents of the invention. Spermidine (17) can be converted to a bis-guanidinium derivative (18), or alternately a guanidinium derivative (19), that can be further conjugated with a ligand (L) to generate (20). Any of compounds 17-20 can be complexed with a compound of interest, such as a nucleic acid or siNA construct, to generate a composition to facilitate the cellular delivery of the compound of interest. Other polyamines having differing alkyl chain lengths and nitrogen content, such as spermine, can be similarly used to generate a variety of diamino, bis-guanidinium, guanidinium, and ligand derivatized complexes.

FIG. 6 shows non-limiting examples of cationic delivery reagents of the invention. Tris-(2-aminoethyl)amine (TREN) (21) can be converted to a tri-guanidinium derivative (22), bis-guanidinium derivative (24), or alternately guanidinium derivative (23). Compounds (23) and (24) can be further conjugated with a ligand (L) to generate compounds (25) and (26), and compound (25) can be further conjugated with the same or a different ligand to generate compound (27). Any of compounds 21-27 can be complexed with a compound of interest, such as a nucleic acid or siNA construct, to generate a composition to facilitate the cellular delivery of the compound of interest. Other tris-(aminoaklyl)amines having differing alkyl chain lengths can be similarly used to generate a variety of tri-guanidinium, bis-guanidinium, guanidinium, and ligand derivatized complexes.

FIG. 7 shows a non-limiting example of the synthesis of a sperm idine based conjugate of the invention. Spermine (17) is converted to a bis-guanidinium derivative (18) using di-Boc pyrazole carboxamidine. Compound 18 can comprise free guanidinium groups (R═H) or alternately partially protected guanidinium groups (R=Boc). Compound (18) is then coupled with a ligand (L), for via an amide linkage, to generate compound (28). Compound (28) can be complexed with a compound of interest, such as a nucleic acid or siNA construct, to generate a composition to facilitate the cellular delivery of the compound of interest. Other polyamines having differing alkyl chain lengths and nitrogen content can be similarly used to generate a variety of such polyamine ligand derivatized complexes.

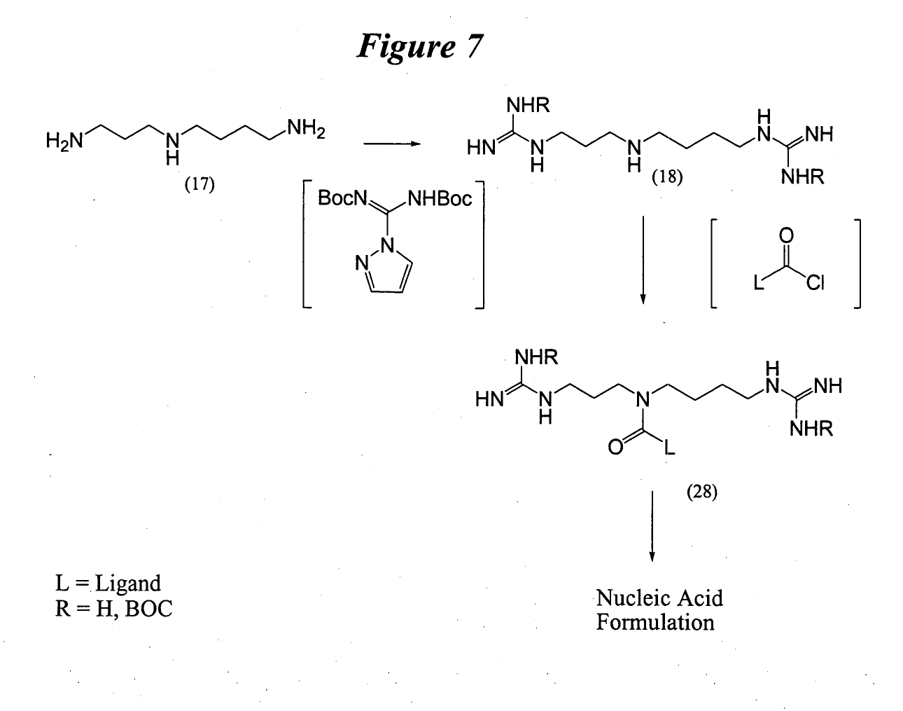

FIG. 8 shows a non-limiting example of the synthesis of an EDTA based conjugate of the invention. A diamine, such as diaminopropane (29), is coupled to a ligand, for example via an amide linkage, for generate compound (30) bearing a free amine. Compound (30) is then coupled with EDTA to generate compound (31), which is then coupled with a polyamine, such as compound (18), to generate compound (32), bearing one, two, or three bis-guanidinium substituents.). Compound (32) can be complexed with a compound of interest, such as a nucleic acid or siNA construct, to generate a composition to facilitate the cellular delivery of the compound of interest. Other polyamines having differing alkyl chain lengths and nitrogen content can be similarly used to generate a variety of such polyamine ligand derivatized complexes.

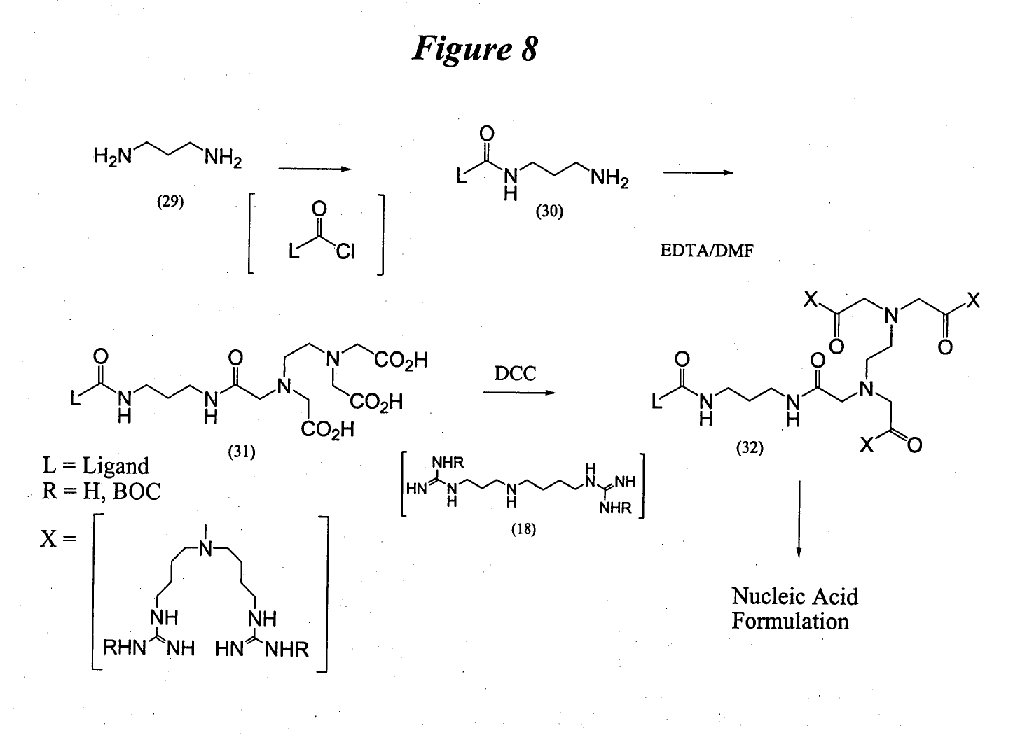

FIG. 9 shows a non-limiting example of the synthesis of a 4-N-(Cholesterol-PEG)-Spermidine conjugate of the invention.

FIG. 10 shows a non-limiting example of the synthesis of a 4-N-(Cholesterol-PEG)-Spermidyl-Bis-guanidine conjugate of the invention.

FIG. 11 shows non-limiting examples of different stabilization chemistries (1-10) that can be used, for example, to stabilize the 3′-end of siNA sequences of the invention, including (1) [3-3′]-inverted deoxyribose; (2) deoxyribonucleotide; (3) [5′-3′]-3′-deoxyribonucleotide; (4) [5′-3′]-ribonucleotide; (5) [5′-3′]-3′-O-methyl ribonucleotide; (6) 3′-glyceryl; (7) [3′-5′]-3′-deoxyribonucleotide; (8) [3′-3′]deoxyribonucleotide; (9) [5′-2′]-deoxyribonucleotide; and (10) [5-3′]-dideoxyribonucleotide. In addition to modified and unmodified backbone chemistries indicated in the figure, these chemistries can be combined with different backbone modifications. In addition, the 2′-deoxy nucleotide shown 5′ to the terminal modifications shown can be another modified or unmodified nucleotide or non-nucleotide.

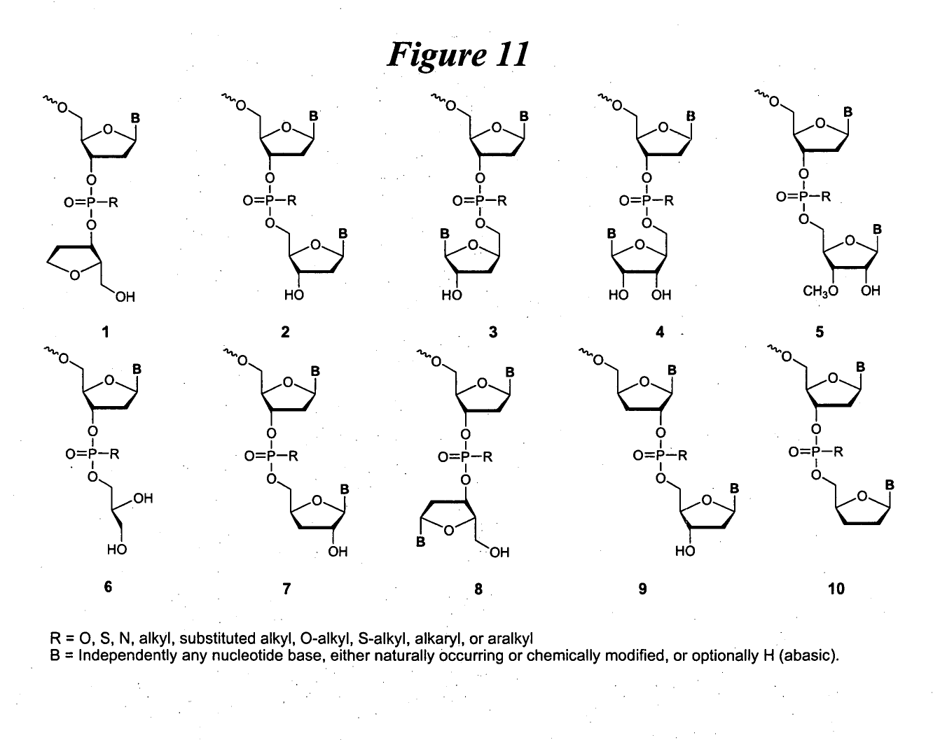

### DETAILED DESCRIPTION OF THE INVENTION

Method of Use

The compounds (e.g. compounds having any for Formulae 1-60 and/or biologically active molecules) of the instant invention can be used to administer pharmaceutical agents, such as biologically active molecules described herein. Pharmaceutical agents prevent, inhibit the occurrence, or treat (alleviate a symptom to some extent, preferably all of the symptoms) of a disease state in a patient.

Generally, the compounds (e.g. compounds having any for Formulae 1-60 and/or biologically active molecules) of the instant invention are introduced by any standard means, with or without stabilizers, buffers, and the like, to form a composition. For use of a liposome delivery mechanism, standard protocols for formation of liposomes can be followed. The compositions of the present invention can also be formulated and used as tablets, capsules or elixirs for oral administration; suppositories for rectal administration; sterile solutions; suspensions for injectable administration; and the like.

The present invention also includes pharmaceutically acceptable formulations of the compounds described above, preferably in combination with the molecule(s) to be delivered. These formulations include salts of the above compounds, e.g., acid addition salts, for example, salts of hydrochloric, hydrobromic, acetic acid, and benzene sulfonic acid.

In one embodiment, the invention features the use of the compounds of the invention in a composition comprising surface-modified liposomes containing poly (ethylene glycol) lipids (PEG-modified, or long-circulating liposomes or stealth liposomes). In another embodiment, the invention features the use of compounds of the invention covalently attached to polyethylene glycol. These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such compositions have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating compositions enhance the pharmacokinetics and pharmacodynamics of therapeutic compounds, such as DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating compositions are also likely to protect drugs from nuclease degradation to a greater extent compared to cationic liposomes, based on their ability to avoid accumulation in metabolically aggressive MPS tissues such as the liver and spleen.

The present invention also includes a composition(s) prepared for storage or administration that includes a pharmaceutically effective amount of the desired compound(s) in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A. R. Gennaro edit. 1985) hereby incorporated by reference herein. For example, preservatives, stabilizers, dyes and flavoring agents can be included in the composition. Examples of such agents include but are not limited to sodium benzoate, sorbic acid and esters of p-hydroxybenzoic acid. In addition, antioxidants and suspending agents can be included in the composition.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer. Furthermore, the compounds of the invention and formulations thereof can be administered to a fetus via administration to the mother of a fetus.

The compounds of the invention and formulations thereof can be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example, sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The compounds of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form will vary depending upon the host treated and the particular mode of administration. Dosage unit forms will generally contain between from about 1 mg to about 500 mg of an active ingredient.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The compounds of the present invention can also be administered to a patient in combination with other therapeutic compounds to increase the overall therapeutic effect. The use of multiple compounds to treat an indication can increase the beneficial effects while reducing the presence of side effects.

Synthesis of Nucleic Acid Molecules

Synthesis of nucleic acids greater than 100 nucleotides in length is difficult using automated methods, and the therapeutic cost of such molecules is prohibitive. In this invention, small nucleic acid motifs “small” refers to nucleic acid motifs no more than 100 nucleotides in length, preferably no more than 80 nucleotides in length, and most preferably no more than 50 nucleotides in length; (e.g., individual siNA oligonucleotide sequences or siNA sequences synthesized in tandem) are preferably used for exogenous delivery. The simple structure of these molecules increases the ability of the nucleic acid to invade targeted regions of protein and/or RNA structure. Exemplary molecules of the instant invention are chemically synthesized, and others can similarly be synthesized.

Oligonucleotides (e.g., certain modified oligonucleotides or portions of oligonucleotides lacking ribonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of oligonucleotides makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 minute coupling step for 2′-O-methylated nucleotides and a 45 second coupling step for 2′-deoxy nucleotides or 2′-deoxy-2′-fluoro nucleotides. Table I outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl residues relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA-based oligonucleotides is performed as follows: the polymer-bound trityl-on oligoribonucleotide is transferred to a 4 mL glass screw top vial and suspended in a solution of 40% aqueous methylamine (1 mL) at 65° C. for 10 minutes. After cooling to −20° C., the supernatant is removed from the polymer support. The support is washed three times with 1.0 mL of EtOH:MeCN:H2O/3:1:1, vortexed and the supernatant is then added to the first supernatant. The combined supernatants, containing the oligoribonucleotide, are dried to a white powder.

The method of synthesis used for RNA including certain siNA molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 minute coupling step for alkylsilyl protected nucleotides and a 2.5 minute coupling step for 2′-O-methylated nucleotides. Table I outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl residues relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo residues relative to polymer-bound 5′-hydroxyl. Average coupling yields on the 394 Applied Biosystems, Inc. synthesizer, determined by colorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM I₂, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection and purification of the siNA can be performed as is generally described in Usman et al., U.S. Pat. No. 5,831,071, U.S. Pat. No. 6,353,098, U.S. Pat. No. 6,437,117, and Bellon et al., U.S. Pat. No. 6,054,576, U.S. Pat. No. 6,162,909, U.S. Pat. No. 6,303,773, or Scaringe supra, incorporated by reference herein in their entireties. Additionally, deprotection conditions can be modified to provide the best possible yield and purity of siNA constructs. For example, applicant has observed that oligonucleotides comprising 2′-deoxy-2′-fluoro nucleotides can degrade under inappropriate deprotection conditions. Such oligonucleotides are deprotected using aqueous methylamine at about 35° C. for 30 minutes. If the 2′-deoxy-2′-fluoro containing oligonucleotide also comprises ribonucleotides, after deprotection with aqueous methylamine at about 35° C. for 30 minutes, TEA-HF is added and the reaction maintained at about 65° C. for an additional 15 minutes.

Alternatively, for the one-pot protocol, the polymer-bound trityl-on oligoribonucleotide is transferred to a 4 mL glass screw top vial and suspended in a solution of 33% ethanolic methylamine/DMSO: 1/1 (0.8 mL) at 65° C. for 15 minutes. The vial is brought to room temperature. TEA.3HF (0.1 mL) is added and the vial is heated at 65° C. for 15 minutes. The sample is cooled at −20° C. and then quenched with 1.5 M NH₄HCO₃.

For purification of the trityl-on oligomers, the quenched NH₄HCO₃ solution is loaded onto a C-18 containing cartridge that had been prewashed with acetonitrile followed by 50 mM TEAA. After washing the loaded cartridge with water, the RNA is detritylated with 0.5% TFA for 13 minutes. The cartridge is then washed again with water, salt exchanged with 1 M NaCl and washed with water again. The oligonucleotide is then eluted with 30% acetonitrile.

The average stepwise coupling yields are typically>98% (Wincott et al., 1995 Nucleic Acids Res. 23, 2677-2684). Those of ordinary skill in the art will recognize that the scale of synthesis can be adapted to be larger or smaller than the example described above including but not limited to 96-well format.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

The nucleic acid molecules (e.g. siNA molecules) of the invention can also be synthesized via a tandem synthesis methodology, wherein both siNA strands are synthesized as a single contiguous oligonucleotide fragment or strand separated by a cleavable linker which is subsequently cleaved to provide separate siNA fragments or strands that hybridize and permit purification of the siNA duplex (see McSwiggen et al., U.S. Ser. No. (10/444,853), filed May 23, 2003). The linker can be a polynucleotide linker or a non-nucleotide linker. The tandem synthesis of siNA as described herein can be readily adapted to both multiwell/multiplate synthesis platforms such as 96 well or similarly larger multi-well platforms. The tandem synthesis of siNA as described herein can also be readily adapted to large scale synthesis platforms employing batch reactors, synthesis columns and the like.

A nucleic acid molecule (e.g. siNA molecule) can also be assembled from two distinct nucleic acid strands or fragments wherein one fragment includes the sense region and the second fragment includes the antisense region of the RNA molecule.

The nucleic acid molecules of the present invention can be modified extensively to enhance stability by modification with nuclease resistant groups, for example, 2′-amino, 2′-C-allyl, 2′-fluoro, 2′-O-methyl, 2′-H (for a review see Usman and Cedergren, 1992, TIBS 17, 34; Usman et al., 1994, Nucleic Acids Symp. Ser. 31, 163). siNA constructs can be purified by gel electrophoresis using general methods or can be purified by high pressure liquid chromatography (HPLC; see Wincott et al., supra, the totality of which is hereby incorporated herein by reference) and re-suspended in water.

Optimizing Activity of the Nucleic Acid Molecules of the Invention.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Earnshaw and Gait, 1998, Biopolymers (Nucleic acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid molecules of the instant invention.

While chemical modification of oligonucleotide internucleotide linkages with phosphorothioate, phosphorothioate, and/or 5′-methylphosphonate linkages improves stability, too many of these modifications may cause some toxicity. Therefore, when designing nucleic acid molecules the amount of these internucleotide linkages should be minimized. Without being bound by any particular theory, the reduction in the concentration of these linkages should lower toxicity resulting in increased efficacy and higher specificity of these molecules.

Nucleic acid molecules having chemical modifications that maintain or enhance activity are provided. Such nucleic acid is also generally more resistant to nucleases than unmodified nucleic acid. Thus, in a cell and/or in vivo the activity can not be significantly lowered. Therapeutic nucleic acid molecules (e.g., enzymatic nucleic acid molecules and antisense nucleic acid molecules) delivered exogenously are optimally stable within cells until translation of the target RNA has been inhibited long enough to reduce the levels of the undesirable protein. This period of time varies between hours to days depending upon the disease state. The nucleic acid molecules should be resistant to nucleases in order to function as effective intracellular therapeutic agents. Improvements in the chemical synthesis of RNA and DNA (Wincott et al., 1995 Nucleic Acids Res. 23, 2677; Caruthers et al., 1992, Methods in Enzymology 211, 3-19 (incorporated by reference herein) have expanded the ability to modify nucleic acid molecules by introducing nucleotide modifications to enhance their nuclease stability as described above.

Use of the nucleic acid-based molecules of the invention can lead to better treatment of the disease progression by affording the possibility of combination therapies (e.g., multiple antisense or enzymatic nucleic acid molecules targeted to different genes, nucleic acid molecules coupled with known small molecule inhibitors, or intermittent treatment with combinations of molecules (including different motifs) and/or other chemical or biological molecules). The treatment of patients with nucleic acid molecules can also include combinations of different types of nucleic acid molecules.

In another embodiment, nucleic acid molecules having chemical modifications that maintain or enhance biologic activity are provided. Such nucleic acids are also generally more resistant to nucleases than unmodified nucleic acid. Thus, in a cell and/or in vivo the activity of the nucleic acid can not be significantly lowered. As exemplified herein such enzymatic nucleic acids are useful in a cell and/or in vivo even if activity over all is reduced 10 fold (Burgin et al., 1996, Biochemistry, 35, 14090). Such nucleic acids herein are said to “maintain” the activity of an all RNA or all DNA nucleic acid molecule (e.g. siNA, antisense, or enzymatic nucleic acid molecule).

In another aspect the nucleic acid molecules comprise a 5′ and/or a 3′-cap structure.

In another embodiment the 3′-cap includes, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

In one embodiment, the invention features modified nucleic acid molecules with phosphate backbone modifications comprising one or more phosphorothioate, phosphorodithioate, methylphosphonate, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39. These references are hereby incorporated by reference herein.

In connection with 2′-modified nucleotides as described for the invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., WO 98/28317, respectively, which are both incorporated by reference in their entireties.

Various modifications to nucleic acid (e.g., siNA, antisense and ribozyme) structure can be made to enhance the utility of these molecules. For example, such modifications can enhance shelf-life, half-life in vitro, stability, and ease of introduction of such oligonucleotides to the target site, including e.g., enhancing penetration of cellular membranes and conferring the ability to recognize and bind to targeted cells.

Use of these molecules can lead to better treatment of disease progression by affording the possibility of combination therapies (e.g., multiple nucleic acid molecules targeted to different genes, nucleic acid molecules coupled with known small molecule inhibitors, or intermittent treatment with combinations of nucleic acid molecules (e.g. siNA, antisense, ribozymes, aptamers etc.) and/or other chemical or biological molecules). The treatment of patients with nucleic acid molecules can also include combinations of different types of nucleic acid molecules. Therapies can be devised which include a mixture of nucleic acid molecules (e.g. siNA, antisense, ribozymes, aptamers etc), to one or more targets to alleviate symptoms of a disease.

Indications

Particular disease states that can be treated using compounds and compositions of the invention include, but are not limited to, cancers and cancerous conditions such as breast, lung, prostate, colorectal, brain, esophageal, stomach, bladder, pancreatic, cervical, hepatocellular, head and neck, and ovarian cancer, melanoma, lymphoma, glioma, multidrug resistant cancers; ocular conditions such as macular degeneration and diabetic retinopathy, and/or viral infections including HIV, HBV, HCV, CMV, RSV, HSV, poliovirus, influenza, rhinovirus, west nile virus, severe acute respiratory syndrome (SARS) virus, Ebola virus, foot and mouth virus, papilloma virus, and/or SARS virus infection.

The molecules of the invention can be used in conjunction with other known methods, therapies, or drugs. For example, the use of monoclonal antibodies (eg; mAb IMC C225, mAB ABX-EGF) treatment, tyrosine kinase inhibitors (TKIs), for example OSI-774 and ZD1839, chemotherapy, and/or radiation therapy, are all non-limiting examples of a methods that can be combined with or used in conjunction with the compounds of the instant invention. Common chemotherapies that can be combined with nucleic acid molecules of the instant invention include various combinations of cytotoxic drugs to kill the cancer cells. These drugs include, but are not limited to, paclitaxel (Taxol), docetaxel, cisplatin, methotrexate, cyclophosphamide, doxorubin, fluorouracil carboplatin, edatrexate, gemcitabine, vinorelbine etc. Those skilled in the art will recognize that other drug compounds and therapies can be similarly be readily combined with the compounds of the instant invention are hence within the scope of the instant invention.

### EXAMPLES

The following are non-limiting examples showing the selection, isolation, synthesis and activity compositions of the instant invention.

### Example 1

### Synthesis of Cationic Polymers

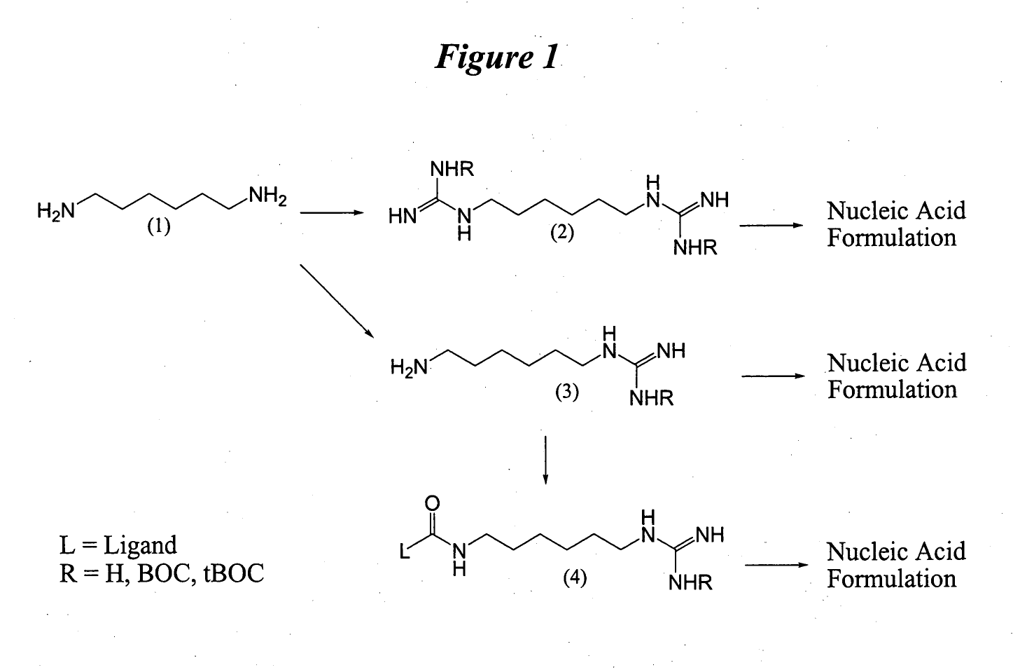

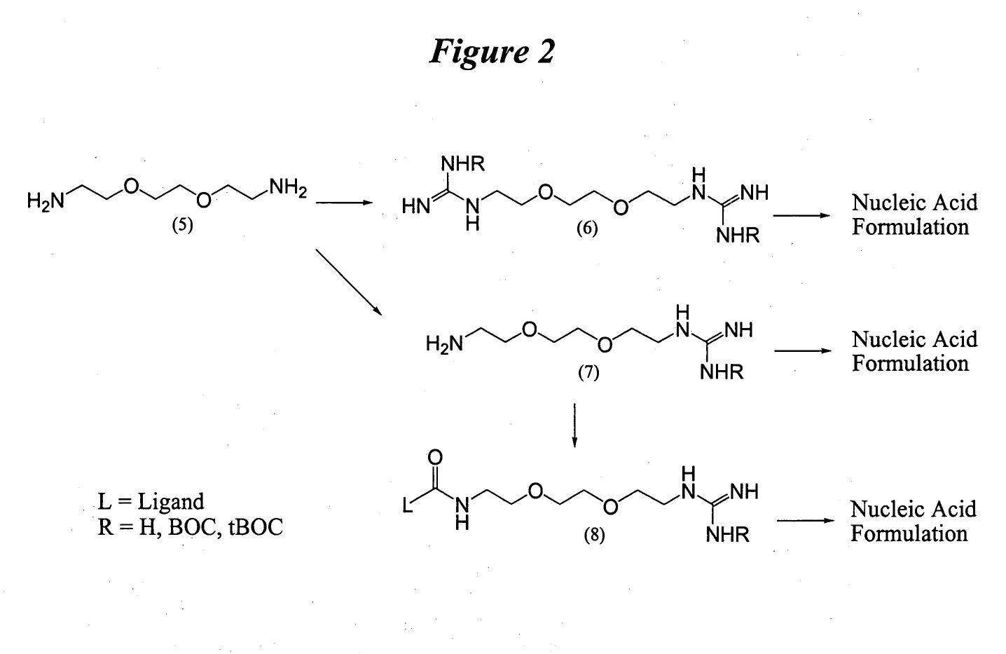

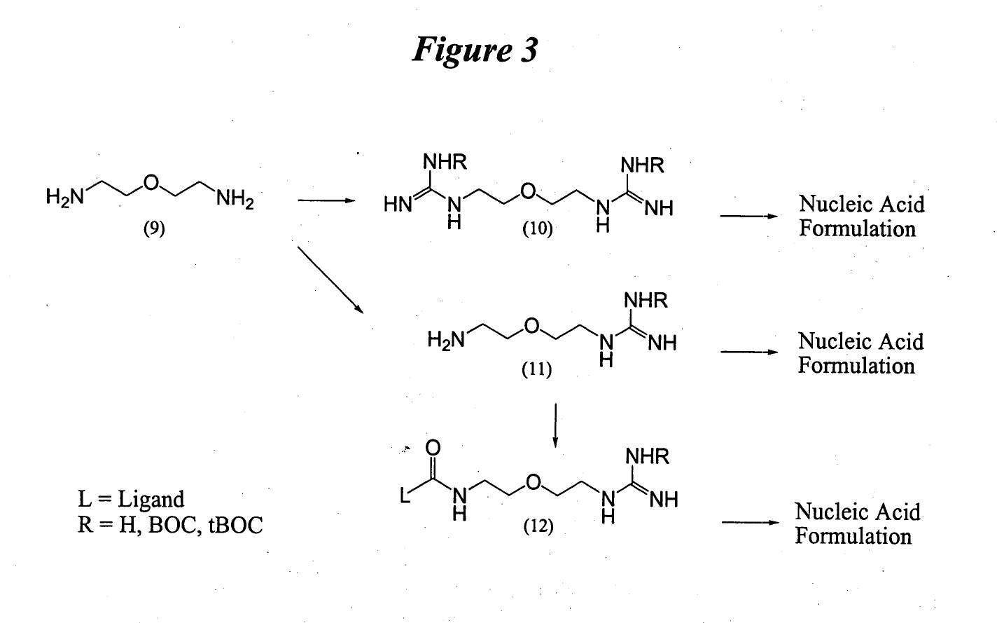

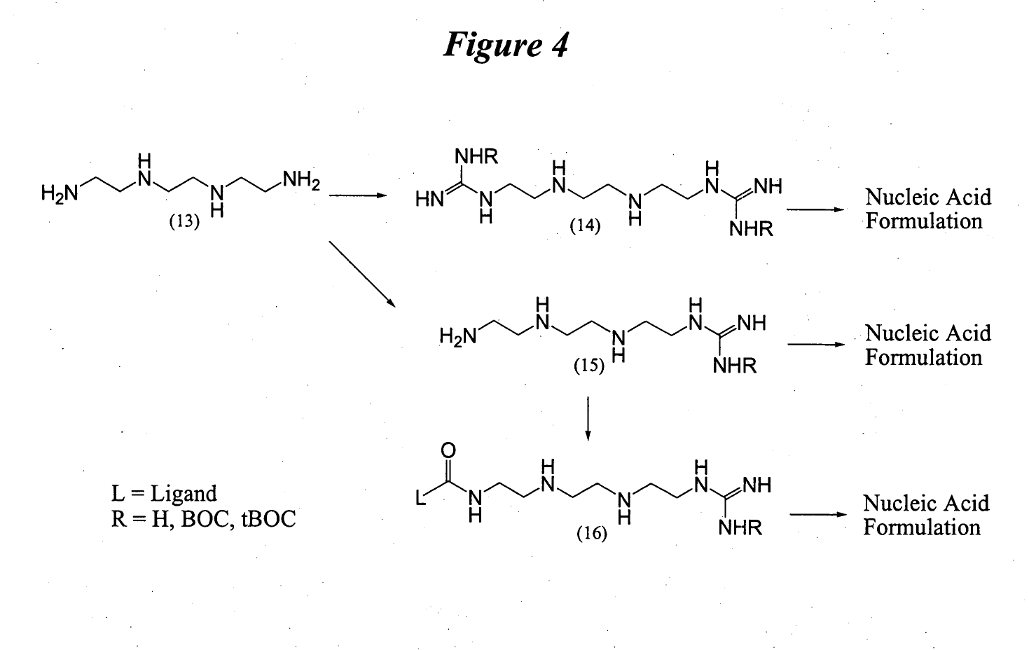

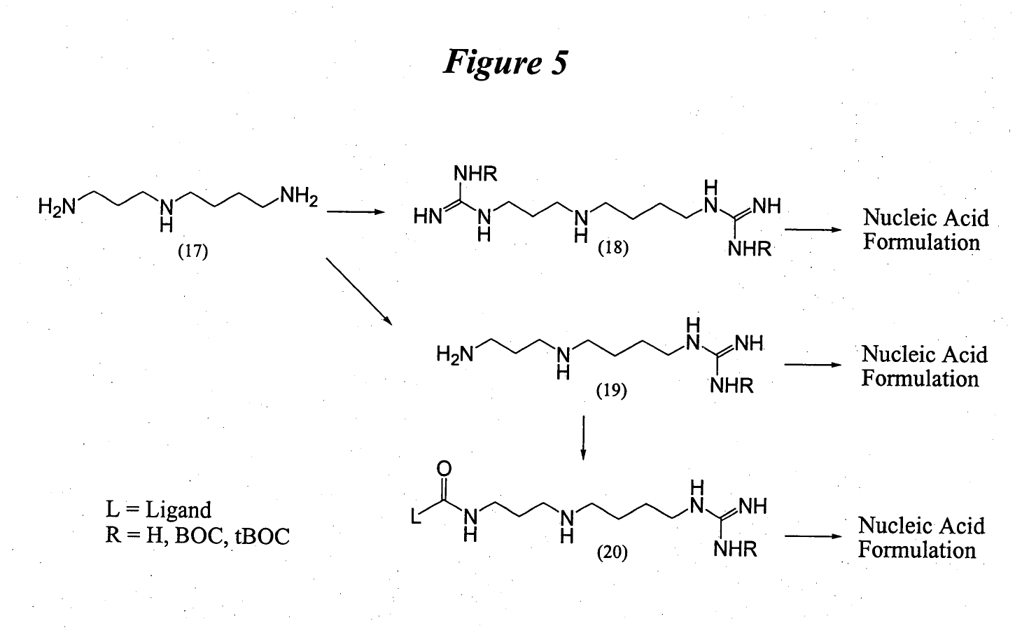

### Generalized Synthesis of Bis-Guanidinium Compounds; e.g. Compounds (2), (6), (10), (14), (18), (24) from FIGS. 1-6

To a stirred solution of diamine (1), (5), (9), (13), or (17) or triamine (21) in 1,2-dicholoroethane or other suitable solvent is added N,N′-bis(tert-butoxycarbonyl)-1H-pyrazole-1-carboxamidine (2.0-2.2 equivalents to diamine/triamine). After stirring at room temperature for 24 h, the reaction mixture is concentrated on a rotary evaporator. The resulting solid is applied to a silica gel column and a suitable gradient, such as hexanes/dichloromethane/triethylamine (e.g. 80:15:5) is applied, appropriate fractions are collected and evaporated to yield N,N′-bis(tert-butoxycarbonyl) protected bis-guanidinium intermediates. These compounds are then suspended in anhydrous methanol a solution of 4.0 M hydrogen chloride in 1,4-dioxane is added and the resulting gas is liberated from the reaction. The resulting solution is stirred at 40° C. overnight and is then concentrated under vacuum to yield a solid, which is reconstituted in anhydrous methanol. Bis-guanidinium compounds (2), (6), (10), (14), (18), or (24) are then obtained by crystallization, for example in dichloromethane/methanol.

### Generalized Synthesis of Guanidinium Compounds; e.g. Compounds (3), (7), (11), (15), (19), (23) from FIGS. 1-6

To a stirred solution of diamine (1), (5), (9), (13), or (17) in 1,2-dicholoroethane or other suitable solvent cooled to 0 degrees C. is added N,N′-bis(tert-butoxycarbonyl)-1H-pyrazole-1-carboxamidine (1.1 equivalents to diamine) dropwise via syringe. The reaction is gradually allowed to warm to room temperature while stirring. After stirring at room temperature for 24 h, the reaction mixture is concentrated on a rotary evaporator. The resulting solid is applied to a silica gel column and a suitable gradient, such as hexanes/dichloromethane/triethylamine (e.g. 80:15:5) is applied, appropriate fractions are collected and evaporated to yield N,N′-bis(tert-butoxycarbonyl) protected guanidinium intermediates. These compounds are then suspended in anhydrous methanol a solution of 4.0 M hydrogen chloride in 1,4-dioxane is added and the resulting gas is liberated from the reaction. The resulting solution is stirred at 40° C. overnight and is then concentrated under vacuum to yield a solid, which is reconstituted in anhydrous methanol. Guanidinium compounds (3), (7), (11), (15), (19), or (23) are then obtained by crystallization, for example in dichloromethane/methanol. These compounds or their corresponding bis(tert-butoxycarbonyl) protected intermediates can be used as precursors to ligand conjugated guanidinium compounds (4), (8), (12), (16), (20), (25), (26), or (27) from FIGS. 1-6. Standard coupling chemistries and linkers as are known in the art can be used to couple ligands (e.g. cholesterol, galactose, galactosamine, peptides etc.) to such guanidinium compounds.

### Synthesis of 1,6-bis-guanidinium hexane (2) FIG. 1

To a stirred solution of 1,6-diaminohexane (1) (0.465 g, 4.00 mmol) in 40 mL of 1,2-dichloroethane was added N,N′-bis(tert-butoxycarbonyl)-1H-pyrazole-1-carboxamidine (2.73 g, 8.80 mmol). After stirring at room temperature for 24 h, the reaction mixture was concentrated on a rotary evaporator. The resulting solid was applied on a silica gel column and eluted with hexanes/methylene chloride/triethylamine (80:15:5) to afford a white foam (2.40 g, 100%). ¹H NMR (CDCl₃) □ 11.5 (br, 2H), 8.30 (br, 2H), 3.41 (dt, J₁=6.8 Hz, J₂=7.2 Hz, 4H), 1.60 (m, 4H), 1.50 (s, 18H), 1.49 (s, 18H), 1.41 (m, 4H). ¹³C NMR (CDCl₃) □ 163.8, 156.3, 153.5, 83.2, 79.4, 41.0, 29.1, 28.5, 28.3, 26.8. To the suspension of the above product in 10 mL of anhydrous methanol was added 10 mL of 4.0 M hydrogen chloride solution in 1,4-dioxane. Gas evolution took place immediately. The resulting orange solution was stirred at 40° C. overnight. Concentration under vacuum resulted in brown solid, which was reconstituted in anhydrous methanol. The process was repeated twice, and white crystals (1.10 g, 100%) were precipitated upon the addition of methylene chloride to a methanol. ¹H NMR (CD₃OD) □ 3.38-3.05 (m, 4H), 1.73-1.55 (m, 4H), 1.55-1.37 (m, 4H). ¹³C NMR (CD₃OD) □ 157.4, 41.3, 28.5, 26.1. MS (m/e): 201 (M+1, 100%).

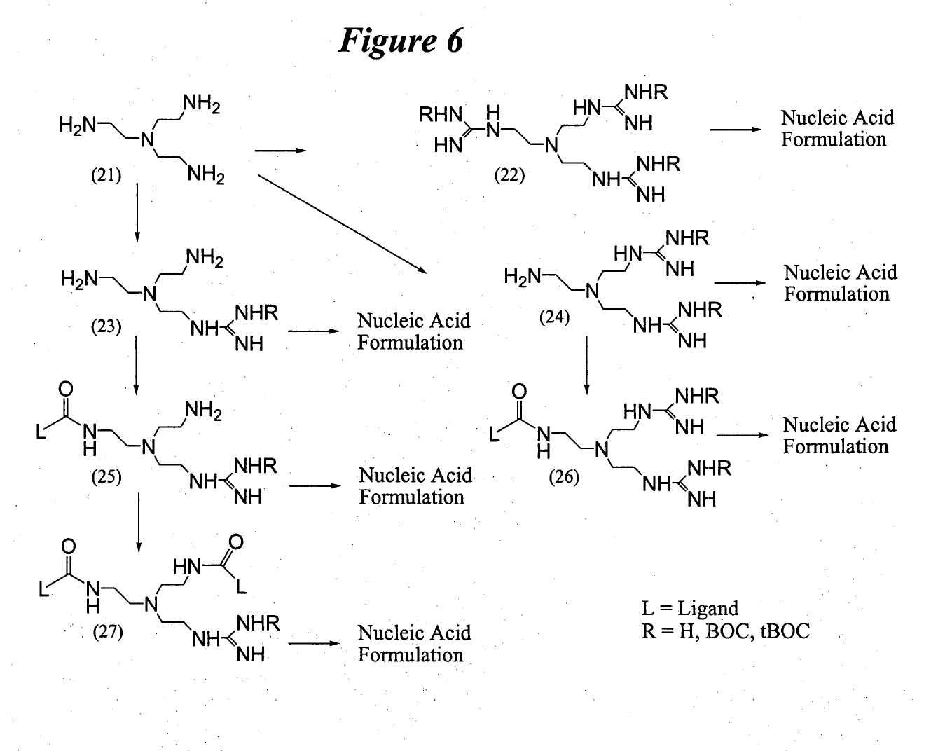

### Synthesis of Tris(2-gaunidinium-ethyl)amine (22) R═H, FIG. 6

Tris(2-aminoethyl)amine (21) (1.50 mL, 10 mmol) was co-evaporated with 1,4-dioxane (2×10 mL), then dissolved in anhydrous 1,4-dioxane. N-tert-butoxycarbonyl-1H-pyrazole-1-carboxamidine (7.56 g, 36 mmol) and TEA (5.0 mL, 36 mmol) were added. The reaction mixture was stirred and heated under Argon overnight. The resulting which precipitate was filtered out, washed with dioxane, and dried under high vacuum overnight to give 1.48 g (26%) of product (22, R=BOC) as a white solid. ¹H NMR (DMSO) δ 1.33 (s, 27H), 2.54 (br t, 6H), 3.16 (br t, 6H); ¹³C NMR (DMSO) δ 33.68 (CH3-Boc), 58.35 (C-1), 71.77 (C-2), 81.28 (C-Boc), 168.69(CO-Boc); ES-MS: 573.4 (+Q1). The white solid (22, R=BOC, 0.5 g, 0.87 mmol) was dissolved in methanol (5 mL) and HCl in dioxane (4M, 5 mL) was added slowly. The reaction mixture was stirred and heated at 50° C. overnight. The resulting white precipitate was then collected and washed with dichloromethane and dried under high vacuum to give 0.418 g (quantitative) of product (22, R═H) as a white solid. ¹H NMR (D₂O) δ 3.38 (t, J=6.0 Hz, 6H), 3.55 (t, J=6.0 Hz, 6H); ¹³C NMR (D₂O) δ 36.19 (C-1), 52.19 (C-2), 157.07 (C-guanidine); ES-MS: 273.2 (+Q1).

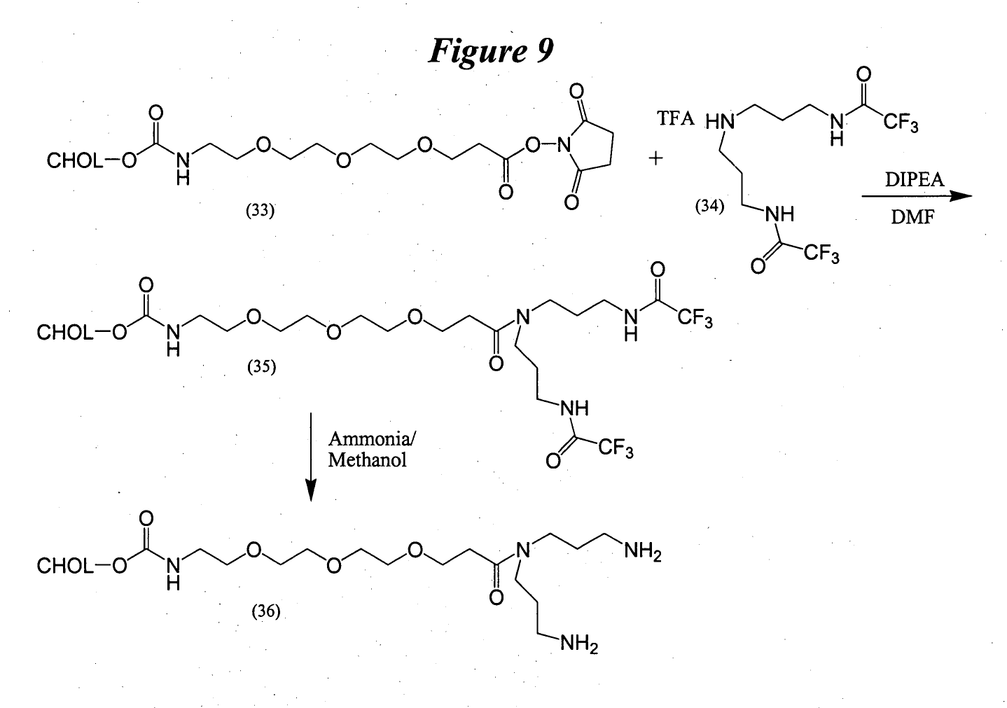

### 4-N-(Cholesterol-PEG)-Spermidine (36), FIG. 9

Cholesterol-PEG-NHS ester (33), (0.50 g, 0.68 mmol) was dissolved in anhydrous DMF (5 mL). The 1,8-di-trifluoroacetyl-spermidine TFA salt (34), (0.40 g, 1.3 eq.) and DIPEA (0.30 mL, 2.5 eq.) were added. The reaction mixture was stirred at room temperature overnight. DMF was removed by rotary evaporation under reduced pressure. The residue obtained was then dissolved in dichloromethane (50 mL) and washed with sodium bicarbonate (5%, 2×50 mL). The organic layer was dried with sodium sulfate, and evaporated to dryness. The residue was chromatographed on silica gel (3% methanol/DCM) to give 0.47 g (72%) of product (35). ES-MS: 953.9 (+Q1); 19F NMR δ −95.36, −95.03 (F-trifluoroacetyl). The product (35) obtained from last step was dissolved in methanol (5 mL). Ammonia (28%, 2 mL) was added dropwise. The reaction mixture was sealed and kept in a shaker at 50° C. overnight. The ammonia was evaporated. The residue obtained was co-evaporated with methanol twice, and lyophilized from water to give 0.385 g of product (36) as a white solid. ES-MS: 762.0 (+Q1).

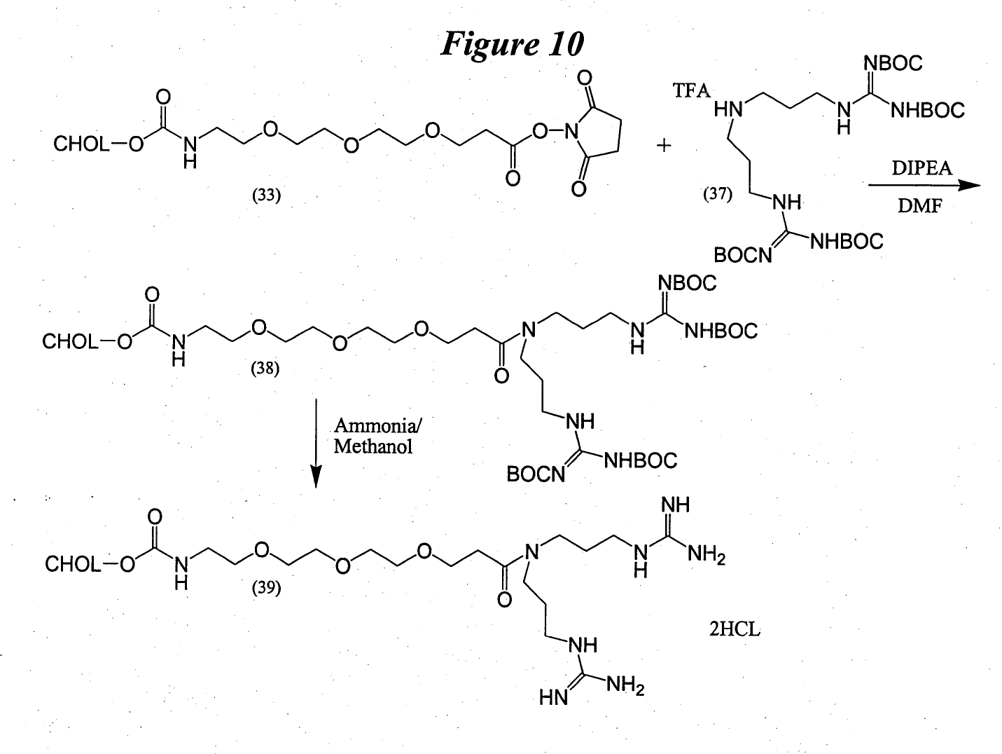

### 4-N-(Cholesterol-PEG)-Spermidyl-Bis-guanidine (39), FIG. 10

The Cholesterol-PEG-NHS ester (33) (0.79 g, 1.08 mmol) was dissolved in anhydrous dichloromethane (10 mL). The 1,8-di-(N,N′-bis-Boc-guanidinium)-permidine (37) (0.885 g, 1.3 eq.) and DIPEA (0.47 mL, 2.5 eq.) were added. The reaction mixture was stirred at room temperature overnight, and then poured into sodium bicarbonate (5% aqueous., 80 mL). The product was extracted with dichloromethane (80 mL). The organic layer was washed with sodium bicarbonate (5%) once, dried with sodium sulfate, and concentrated. The resulting residue was chromatographed on silica gel (50% ethyl acetate/DCM) to give 0.96 g (72%) of product (39) as a white foam. ES-MS: 1246.2 (+Q1).

### Example 2

### Formulation of Polycationic Complexes with siNA

Preparation of Cationic Amine Complexes of siNA

A siNA molecule, such as a siNA duplex, is complexed with a cationic compound based upon charge ratio. The complex can be formulated with different charge ratios by using equivalents of nucleic acid to cation to generate a formulation with a net positive charge (e.g. excess cation to nucleic acid), a neutral charge, or a net negative charge (e.g. excess nucleic acid to cation). The cation can be titrated into a solution of nucleic acid or the nucleic acid can be titrated into a solution of the cationic compound. In a non-limiting example, a siNA duplex comprising sequence (sense strand=5′-fluorescein-ugugcacuucgcuucaccuuu-3′ where a, g, c and u are all ribonucleotides (SEQ ID No: 1)/antisense strand=5′-AGGuGAAGcGAAGuGcAcATsT wherein A and G are 2′-O-methyl nucleotides and u and c are 2′-deoxy-2′-fluoro nucleotides (SEQ ID No: 2)) was obtained in HPLC purified form and dissolved in sterile Milli-Q water to a concentration of 895 uM (approximately 15 mg siNA/ml water). Because there are 42 phosphates per mole of duplex siNA, the net polyanion charge of the duplex is calculated at 37.6 mM in this solution. A 100 uL aliquot of the siNA solution contains 3.8 micromoles of phosphate anion. Two equivalents of cationic lipid, (compound 2, FIG. 1) were added to this solution (as 75 microliters of a 100 mM stock cationic lipid solution). The resulting solution was analyzed by strong anion exchange chromatography for concentration and purity and ion-pairing reverse phase chromatography was used to assay for duplex stability. The solutions were used in a cell culture assay to screen for efficacy of knockdown for mRNA message against HCV virus as described in Example 8 below. In all cases, the polycationic complex with siRNA was found to be intact, full length duplex siRNA and efficacious in the HCV replicon assay. The solution was analyzed for a two week period for solution stability and no changes in concentration or degradation of nucleic acid was noted.

Preparation of Cationic Amine Stock Solutions:

All organic amines and bis-amines were added to water to obtain a 100 millimolar solution and 1N HCl was added dropwise until a pH of 7.1 was obtained. The resulting solution was filtered to 0.2 micron absolute using cell culture grade disposable filters prior to use. The solutions were stored at 5-8 C prior to use. All solutions remained free of precipitates during storage.

Characterization of Polycationic Complexes with Nucleic Acids:

Additional instrumental techniques performed to characterize the cationic complexes included static light scattering and size exclusion chromatography using a Wyatt Technologies miniDawn detector with additional QELS hardware and Astra software (Wyatt Tecnologies, Santa Barbera, Calif.). The size exclusion chromatography was performed using a TosoHaas TSK-gel SWx1 column (4 mm×300 mm) and Agilent 1100 HPLC hardware including binary pump G1312A and RID detector G1362A plus Chemstation software A.08.03. This instrument can detect and quantitate hydrodynamic size of the cationic nucleic acid complexes and provide information on extinction coefficients of the nucleic acid component and molecular weight information of the complex through use of Zimm/Rouse equations for light scattering. A Brookhaven Instrument Corporation ZetaPALS dynamic light scattering instrument was used to measure size distributions of the cationic nucleic acid complexes and characterize the zeta-potential of these complexes. The data collected for all complexes made to date shows a predominant fraction of small monomeric particles for these complexes. A large shift in zeta potential towards positive numbers was observed for all nucleic acid complexes containing cationic amines. The starting siRNA duplex material had a negative zeta potential as is always observed for the polyanionic nucleic acids. A positive zeta-potential is a strong indication that the cationic amine has complexed the nucleic acids and created a new particle with a different net charge than the starting material.

### Example 3

### Formulation of Lipoplex Complexes with Nucleic Acids

Preparation of Lipoplex with Polycationic Amines and Neutral Lipid:

The cationic compounds of the invention (e.g. compounds having any of Formulae 1-60) can be formulated into a lipoplex comprising a cationic component, a lipid component, and a biologically active molecule component (e.g. siNA). The formation of a lipoplex can lead to improved pharmacokinetic properties such as increased half life and increased serum stability of biologically active molecules to be delivered to relevant cells and tissues. In a non-limiting example, a standard neutral phosphatidylethanolamine lipid was purchased from Avanti Polar Lipids as a 10 mg/mL solution in chloroform (Avanti Cat. No. 850402, 1,2-Diphytanoyl-sn-Glycero-3-Phosphoethanolamine, F.W. 804.19). A cationic amine conjugated to cholesterol via a tetraethylene glycol ether linkage (compound 36, FIG. 9) was prepared as described herein. 550 uL of the Cholesterol conjugate at 20 mg/mL in chloroform was added to 900 uL of DPhPE neutral lipid at 10 mg/mL and the solution was evaporated to dryness on a Buichi rotary evaporator with a water bath temperature of 25C. The flask containing the film of lipids in a 1:1 stoichiometric ratio was placed on a vacuum manifold and pumped overnight with a belt driven rotary vane vacuum pump to remove residual chloroform solvent. The dry lipid film was re-hydrated for 2 hours with the addition of 2 mL of sterile water and brief periods of sonication (2×10 minutes each period to prevent overheating of the lipoplex formulation). The sonication was followed by particle size measurement with a Brookhaven Instruments Zeta-PALLS instrument. The light scattering data showed the presence of a monodisperse particle with an effective diameter of 107 nm.

Preparation of Cationic Amine Lipoplexes of siRNA:

Duplex siNA (stab 9/10 active to site 1580 HBV, sense strand=B UGUGCACUUCGCUUCACCUTT B where B is an inverted deoxy abasic cap SEQ ID No: 3, and antisense strand=AGGUGAAGCGAAGUGCACATsT where s is a phosphorothioate, SEQ ID No: 4) and a matched chemistry inverted duplex control siRNA (stab 9/10 inv ctrl to site 1580 HBV, sense strand=B UCCACUUCGCUUCACGUGUTT B where B is an inverted deoxy abasic cap SEQ ID No: 5, and antisense strand=ACACGUGAAGCGAAGUGGATsT where s is a phosphorothioate, SEQ ID No: 6) were obtained in an HPLC purified form and dissolved in sterile Milli-Q water to a concentration of 238 uM (4.0 mg nucleic acid per mL water). There are exactly 42 phosphates per mole of duplex siNA for each duplex resulting in a net polyanion charge of 10.0 mM for these two solutions. A 100 uL aliquot of this siNA solution contains 1.0 micromole of phosphate anion. Two equivalents of cationic lipid were added to this solution as 20 microliters of a 100 mM stock cationic lipid solution.

The siNA to lipoplex charge ratio was titrated between 1:2, 1:3, 1:4, and 1:5 mole equivalents of siNA phosphate to compound (36). These titration experiments resulted in a lipoplex with overall net positive charge in all cases (20% of cationic sites occupied at 1:5 ratio with siNA phosphates to 50% occupation at 1:2 ratio) and these results were confirmed with zeta potential measurement using the BIC zeta-PALLS instrument. All formulations yielded a positive Zeta potential after complexing siNA at the previously described ratios of siNA to lipoplex. To generate a 1 to 2 ratio of siNA to lipoplex required the following concentrations and volume of lipoplex and siNA solutions. For 550 uL of compound (36) at 20 mg/mL in chloroform, 2 amine equivalents in a final volume of 2 mL of water, yielded a lipoplex at 14.5 mM amine concentration. The siNA phosphate solutions used were previously prepared at 8.3 mM after dilution with cationic amine ion-pairing agents.

### Example 4

### Chemical Synthesis and Purification of siNA

siNA molecules can be designed to interact with various sites in the RNA message, for example, target sequences within the RNA sequences described herein. The sequence of one strand of the siNA molecule(s) is complementary to the target site sequences described above. The siNA molecules can be chemically synthesized using methods described herein. Inactive siNA molecules that are used as control sequences can be synthesized by scrambling the sequence of the siNA molecules such that it is not complementary to the target sequence. Generally, siNA constructs can by synthesized using solid phase oligonucleotide synthesis methods as described herein (see for example Usman et al., U.S. Pat. Nos. 5,804,683; 5,831,071; 5,998,203; 6,117,657; 6,353,098; 6,362,323; 6,437,117; 6,469,158; Scaringe et al., U.S. Pat. Nos. 6,111,086; 6,008,400; 6,111,086 all incorporated by reference herein in their entirety).

In a non-limiting example, RNA oligonucleotides are synthesized in a stepwise fashion using the phosphoramidite chemistry as is known in the art. Standard phosphoramidite chemistry involves the use of nucleosides comprising any of 5′-O-dimethoxytrityl, 2′-O-tert-butyldimethylsilyl, 3′-O-2-Cyanoethyl N,N-diisopropylphos-phoroamidite groups, and exocyclic amine protecting groups (e.g. N6-benzoyl adenosine, N4 acetyl cytidine, and N2-isobutyryl guanosine). Alternately, 2′-O-Silyl Ethers can be used in conjunction with acid-labile 2′-O-orthoester protecting groups in the synthesis of RNA as described by Scaringe supra. Differing 2′ chemistries can require different protecting groups, for example 2′-deoxy-2′-amino nucleosides can utilize N-phthaloyl protection as described by Usman et al., U.S. Pat. No. 5,631,360, incorporated by reference herein in its entirety).

During solid phase synthesis, each nucleotide is added sequentially (3′- to 5′-direction) to the solid support-bound oligonucleotide. The first nucleoside at the 3′-end of the chain is covalently attached to a solid support (e.g., controlled pore glass or polystyrene) using various linkers. The nucleotide precursor, a ribonucleoside phosphoramidite, and activator are combined resulting in the coupling of the second nucleoside phosphoramidite onto the 5′-end of the first nucleoside. The support is then washed and any unreacted 5′-hydroxyl groups are capped with a capping reagent such as acetic anhydride to yield inactive 5′-acetyl moieties. The trivalent phosphorus linkage is then oxidized to a more stable phosphate linkage. At the end of the nucleotide addition cycle, the 5′-O-protecting group is cleaved under suitable conditions (e.g., acidic conditions for trityl-based groups and Fluoride for silyl-based groups). The cycle is repeated for each subsequent nucleotide.

Modification of synthesis conditions can be used to optimize coupling efficiency, for example by using differing coupling times, differing reagent/phosphoramidite concentrations, differing contact times, differing solid supports and solid support linker chemistries depending on the particular chemical composition of the siNA to be synthesized. Deprotection and purification of the siNA can be performed as is generally described in Usman et al., U.S. Pat. No. 5,831,071, U.S. Pat. No. 6,353,098, U.S. Pat. No. 6,437,117, and Bellon et al., U.S. Pat. No. 6,054,576, U.S. Pat. No. 6,162,909, U.S. Pat. No. 6,303,773, or Scaringe supra, incorporated by reference herein in their entireties. Additionally, deprotection conditions can be modified to provide the best possible yield and purity of siNA constructs. For example, applicant has observed that oligonucleotides comprising 2′-deoxy-2′-fluoro nucleotides can degrade under inappropriate deprotection conditions. Such oligonucleotides are deprotected using aqueous methylamine at about 35° C. for 30 minutes. If the 2′-deoxy-2′-fluoro containing oligonucleotide also comprises ribonucleotides, after deprotection with aqueous methylamine at about 35° C. for 30 minutes, TEA-HF is added and the reaction maintained at about 65° C. for an additional 15 minutes.

### Example 5

### Nucleic Acid Inhibition of Target RNA In Vivo

siNA molecules targeted to the target RNA are designed, synthesized, and formulated with polycationic delivery compounds as described above. These complexed nucleic acid molecules can be tested for cleavage activity in vivo, for example, using the following procedure.

Two formats are used to test the efficacy of siNAs targeting a particular gene transcipt. First, the reagents are tested on target expressing cells (e.g., HeLa), to determine the extent of RNA and protein inhibition. siNA reagents are selected against the RNA target. RNA inhibition is measured after delivery of these reagents to cells using formulations of the invention. Relative amounts of target RNA are measured versus actin using real-time PCR monitoring of amplification (eg., ABI 7700 Taqman®). A comparison is made to a mixture of oligonucleotide sequences made to unrelated targets or to a randomized siNA control with the same overall length and chemistry, but with randomly substituted nucleotides at each position. Primary and secondary lead reagents are chosen for the target and optimization performed. After an optimal transfection agent and concentration is chosen, a RNA time-course of inhibition is performed with the lead siNA molecule. In addition, a cell-plating format can be used to determine RNA inhibition.

Delivery of siNA to Cells

Cells (e.g., HeLa) are seeded, for example, at 1×10⁵ cells per well of a six-well dish in EGM-2 (BioWhittaker) the day before transfection. siNA (final concentration, for example 20 nM) and cationic delivery agent (e.g., final concentration 20 g/ml) are complexed in EGM basal media (Biowhittaker) at 37° C. for 30 mins in polystyrene tubes. Following vortexing, the complexed siNA is added to each well and incubated for the times indicated. For initial optimization experiments, cells are seeded, for example, at 1×10³ in 96 well plates and siNA complex added as described. Efficiency of delivery of siNA to cells is determined using a fluorescent siNA complexed with lipid. Cells in 6-well dishes are incubated with siNA for 24 hours, rinsed with PBS and fixed in 2% paraformaldehyde for 15 minutes at room temperature. Uptake of siNA is visualized using a fluorescent microscope.

Taqman and Lightcycler Quantification of mRNA

Total RNA is prepared from cells following siNA delivery, for example, using Qiagen RNA purification kits for 6-well or Rneasy extraction kits for 96-well assays. For Taqman analysis, dual-labeled probes are synthesized with the reporter dye, FAM or JOE, covalently linked at the 5′-end and the quencher dye TAMRA conjugated to the 3′-end. One-step RT-PCR amplifications are performed on, for example, an ABI PRISM 7700 Sequence Detector using 50 μl reactions consisting of 10 μl total RNA, 100 nM forward primer, 900 nM reverse primer, 100 nM probe, 1×TaqMan PCR reaction buffer (PE-Applied Biosystems), 5.5 mM MgCl₂, 300 μM each dATP, dCTP, dGTP, and dTTP, 10 U RNase Inhibitor (Promega), 1.25 U AmpliTaq Gold (PE-Applied Biosystems) and 10 U M-MLV Reverse Transcriptase (Promega). The thermal cycling conditions can consist of 30 min at 48° C., 10 min at 95° C., followed by 40 cycles of 15 sec at 95° C. and 1 min at 60° C. Quantitation of mRNA levels is determined relative to standards generated from serially diluted total cellular RNA (300, 100, 33, 11 ng/rxn) and normalizing to β-actin or GAPDH mRNA in parallel TaqMan reactions. For each gene of interest an upper and lower primer and a fluorescently labeled probe are designed. Real time incorporation of SYBR Green I dye into a specific PCR product can be measured in glass capillary tubes using a lightcyler. A standard curve is generated for each primer pair using control cRNA. Values are represented as relative expression to GAPDH in each sample.

Western Blotting

Nuclear extracts can be prepared using a standard micro preparation technique (see for example Andrews and Faller, 1991, Nucleic Acids Research, 19, 2499). Protein extracts from supernatants are prepared, for example using TCA precipitation. An equal volume of 20% TCA is added to the cell supernatant, incubated on ice for 1 hour and pelleted by centrifugation for 5 minutes. Pellets are washed in acetone, dried and resuspended in water. Cellular protein extracts are run on a 10% Bis-Tris NuPage (nuclear extracts) or 4-12% Tris-Glycine (supernatant extracts) polyacrylamide gel and transferred onto nitro-cellulose membranes. Non-specific binding can be blocked by incubation, for example, with 5% non-fat milk for 1 hour followed by primary antibody for 16 hour at 4° C. Following washes, the secondary antibody is applied, for example (1:10,000 dilution) for 1 hour at room temperature and the signal detected with SuperSignal reagent (Pierce).

### Example 6

### Animal Models

Various animal models can be used to screen formulated siNA constructs in vivo as are known in the art, for example those animal models that are used to evaluate other nucleic acid technologies such as enzymatic nucleic acid molecules (ribozymes) and/or antisense. Such animal models are used to test the efficacy of formulated siNA molecules described herein. In a non-limiting example, siNA molecules that are designed as anti-angiogenic agents can be screened using animal models. There are several animal models available in which to test the anti-angiogenesis effect of nucleic acids of the present invention, such as siNA, directed against genes associated with angiogenesis and/or metastais, such as VEGFR (e.g., VEGFR1, VEGFR2, and VEGFR3) genes. Typically a corneal model has been used to study angiogenesis in rat and rabbit, since recruitment of vessels can easily be followed in this normally avascular tissue (Pandey et al., 1995 Science 268: 567-569). In these models, a small Teflon or Hydron disk pretreated with an angiogenesis factor (e.g. bFGF or VEGF) is inserted into a pocket surgically created in the cornea. Angiogenesis is monitored 3 to 5 days later. siNA molecules directed against VEGFR mRNAs would be delivered in the disk as well, or dropwise to the eye over the time course of the experiment. In another eye model, hypoxia has been shown to cause both increased expression of VEGF and neovascularization in the retina (Pierce et al., 1995 Proc. Natl. Acad. Sci. USA. 92: 905-909; Shweiki et al., 1992J. Clin. Invest. 91: 2235-2243).

Several animal models exist for screening of anti-angiogenic agents. These include corneal vessel formation following corneal injury (Burger et al., 1985 Cornea 4: 35-41; Lepri, et al., 1994 J. Ocular Pharmacol. 10: 273-280; Ormerod et al., 1990 Am. J. Pathol. 137: 1243-1252) or intracorneal growth factor implant (Grant et al., 1993 Diabetologia 36: 282-291; Pandey et al. 1995 supra; Zieche et al., 1992 Lab. Invest. 67: 711-715), vessel growth into Matrigel matrix containing growth factors (Passaniti et al., 1992 supra), female reproductive organ neovascularization following hormonal manipulation (Shweiki et al., 1993 Clin. Invest. 91: 2235-2243), several models involving inhibition of tumor growth in highly vascularized solid tumors (O'Reilly et al., 1994 Cell 79: 315-328; Senger et al., 1993 Cancer and Metas. Rev. 12: 303-324; Takahasi et al., 1994 Cancer Res. 54: 4233-4237; Kim et al., 1993 supra), and transient hypoxia-induced neovascularization in the mouse retina (Pierce et al., 1995 Proc. Natl. Acad. Sci. USA. 92: 905-909).

The cornea model, described in Pandey et al. supra, is the most common and well characterized anti-angiogenic agent efficacy screening model. This model involves an avascular tissue into which vessels are recruited by a stimulating agent (growth factor, thermal or alkalai burn, endotoxin). The corneal model utilizes the intrastromal corneal implantation of a Teflon pellet soaked in a VEGF-Hydron solution to recruit blood vessels toward the pellet, which can be quantitated using standard microscopic and image analysis techniques. To evaluate their anti-angiogenic efficacy, siNA molecules are applied topically to the eye or bound within Hydron on the Teflon pellet itself. This avascular cornea as well as the Matrigel model (described below) provide for low background assays. While the corneal model has been performed extensively in the rabbit, studies in the rat have also been conducted.

The mouse model (Passaniti et al., supra) is a non-tissue model which utilizes Matrigel, an extract of basement membrane (Kleinman et al., 1986) or Millipore® filter disk, which can be impregnated with growth factors and anti-angiogenic agents in a liquid form prior to injection. Upon subcutaneous administration at body temperature, the Matrigel or Millipore® filter disk forms a solid implant. VEGF embedded in the Matrigel or Millipore® filter disk is used to recruit vessels within the matrix of the Matrigel or Millipore® filter disk which can be processed histologically for endothelial cell specific vWF (factor VEGF antigen) immunohistochemistry, Trichrome-Masson stain, or hemoglobin content. Like the cornea, the Matrigel or Millipore® filter disk are avascular; however, it is not tissue. In the Matrigel or Millipore® filter disk model, siNA molecules are administered within the matrix of the Matrigel or Millipore® filter disk to test their anti-angiogenic efficacy. Thus, delivery issues in this model, as with delivery of siNA molecules by Hydron-coated Teflon pellets in the rat cornea model, may be less problematic due to the homogeneous presence of the siNA within the respective matrix.

The Lewis lung carcinoma and B-16 murine melanoma models are well accepted models of primary and metastatic cancer and are used for initial screening of anti-cancer agents. These murine models are not dependent upon the use of immunodeficient mice, are relatively inexpensive, and minimize housing concerns. Both the Lewis lung and B-16 melanoma models involve subcutaneous implantation of approximately 10⁶ tumor cells from metastatically aggressive tumor cell lines (Lewis lung lines 3LL or D122, LLc-LN7; B-16-BL6 melanoma) in C57BL/6J mice. Alternatively, the Lewis lung model can be produced by the surgical implantation of tumor spheres (approximately 0.8 mm in diameter). Metastasis also may be modeled by injecting the tumor cells directly intraveneously. In the Lewis lung model, microscopic metastases can be observed approximately 14 days following implantation with quantifiable macroscopic metastatic tumors developing within 21-25 days. The B-16 melanoma exhibits a similar time course with tumor neovascularization beginning 4 days following implantation. Since both primary and metastatic tumors exist in these models after 21-25 days in the same animal, multiple measurements can be taken as indices of efficacy. Primary tumor volume and growth latency as well as the number of micro- and macroscopic metastatic lung foci or number of animals exhibiting metastases can be quantitated. The percent increase in lifespan can also be measured. Thus, these models would provide suitable primary efficacy assays for screening systemically administered siNA molecules and siNA formulations.

In the Lewis lung and B-16 melanoma models, systemic pharmacotherapy with a wide variety of agents usually begins 1-7 days following tumor implantation/inoculation with either continuous or multiple administration regimens. Concurrent pharmacokinetic studies can be performed to determine whether sufficient tissue levels of siNA can be achieved for pharmacodynamic effect to be expected. Furthermore, primary tumors and secondary lung metastases can be removed and subjected to a variety of in vitro studies (i.e. target RNA reduction).

Ohno-Matsui et al., 2002, Am. J. Pathology, 160, 711-719 describe a model of severe proliferative retinopathy and retinal detachment in mice under inducible expression of vascular endothelial growth factor. In this model, expression of a VEGF transgene results in elevated levels of ocular VEGF that is associated with severe proliferative retinopathy and retinal detachment. Furthermore, Mori et al., 2001, J. Cellular Physiology, 188, 253-263, describe a model of laser induced choroidal neovascularization that can be used in conjunction with intravitreous or subretianl injection of siNA molecules of the invention to evaluate the efficacy of siNA treatment of severe proliferative retinopathy and retinal detachment.

In utilizing these models to assess siNA activity, VEGFR1, VEGFR2, and/or VEGFR3 protein levels can be measured clinically or experimentally by FACS analysis. VEGFR1, VEGFR2, and/or VEGFR3 encoded mRNA levels can be assessed by Northern analysis, RNase-protection, primer extension analysis and/or quantitative RT-PCR. siNA molecules that block VEGFR1, VEGFR2, and/or VEGFR3 protein encoding mRNAs and therefore result in decreased levels of VEGFR1, VEGFR2, and/or VEGFR3 activity by more than 20% in vitro can be identified using the techniques described herein.

### Example 7

### Screening Formulated siNA Constructs for Improved Pharmacokinetics

In a non-limiting example, formulated siNA constructs are screened in vivo for improved pharmacokinetic properties compared to siNA constructs that are not complexed with a delivery agent. Lead siNA molecules are complexed with a delivery agent and the formulated constructs are tested in an appropriate system (e.g human serum for nuclease resistance, shown, or an animal model for PK/delivery parameters). In parallel, the formulated siNA construct is tested for RNAi activity, for example in a cell culture system such as a luciferase reporter assay). Lead siNA formulations are then identified which possess a particular characteristic while maintaining RNAi activity, and can be further modified or optimized and assayed once again. This same approach can be used to identify siNA-conjugate molecules with improved pharmacokinetic profiles, delivery, localized delivery, cellular uptake, and RNAi activity.

### Example 8

### Inhibition of HCV RNA Expression Using Complexed siNA Formulations

Formulated siNA complexes with cationic polymers are tested for efficacy in reducing HCV RNA expression in, for example, Huh7 cells (see, for example, Randall et al., 2003, PNAS USA, 100, 235-240). Cells are plated approximately 24 h before transfection in 96-well plates at 5,000-7,500 cells/well, 100 μl/well, such that at the time of transfection cells are 70-90% confluent. For transfection, annealed siNAs are complexed with a cationic polymer (see Example 2 above) in a volume of 50 μl/well and incubated for 20 minutes at room temperature. The siNA transfection mixtures are added to cells to give a final siNA concentration of 25 nM in a volume of 150 μl. Each siNA transfection mixture is added to 3 wells for triplicate siNA treatments. Cells are incubated at 37° for 24 h in the continued presence of the siNA transfection mixture. At 24 h, RNA is prepared from each well of treated cells. The supernatants with the transfection mixtures are first removed and discarded, then the cells are lysed and RNA prepared from each well. Target gene expression following treatment is evaluated by RT-PCR for the target gene and for a control gene (36B4, an RNA polymerase subunit) for normalization. The triplicate data is averaged and the standard deviations determined for each treatment. Normalized data are graphed and the percent reduction of target mRNA by active siNAs in comparison to their respective inverted control siNAs is determined.

### Example 9

### Formulation of Polycationic Complexes with Nucleic Acid Molecules

Preparation of Cationic Amine Complexes of Nucleic Acid Molecules.

A nucleic acid molecule, such as an enzymatic nucleic acid, antisense, or aptamer, is complexed with a cationic compound based upon charge ratio. The complex can be formulated with different charge ratios by using equivalents of nucleic acid to cation to generate a formulation with a net positive charge (e.g. excess cation to nucleic acid), a neutral charge, or a net negative charge (e.g. excess nucleic acid to cation). The cation can be titrated into a solution of nucleic acid or the nucleic acid can be titrated into a solution of the cationic compound. In a non-limiting example, the nucleic acid molecule is obtained in HPLC purified form and dissolved in sterile Milli-Q water. A stock solution of cationic amine is added in a suitable amount based upon the number of phosphates present in the nucleic acid molecule. For example, to generate a formulation with a net positive charge, an excess molar equivalence is used. The resulting solution is analyzed by strong anion exchange chromatography for concentration and purity. The solutions are then used in a cell culture assays to screen for efficacy of the formulated nucleic acid composition in reducing mRNA levels or protein levels.

Preparation of Cationic Amine Stock Solutions:

All organic amines and bis-amines are added to water to obtain a 100 millimolar solution and 1N HCl was added dropwise until a pH of 7.1 is obtained. The resulting solution is filtered to 0.2 micron absolute using cell culture grade disposable filters prior to use. The solutions are stored at 5-8 C prior to use. All solutions remained free of precipitates during storage.

Characterization of Polycationic Complexes with Nucleic Acids:

Additional instrumental techniques performed to characterize the cationic complexes include static light scattering and size exclusion chromatography using a Wyatt Technologies miniDawn detector with additional QELS hardware and Astra software (Wyatt Tecnologies, Santa Barbera, Calif.). The size exclusion chromatography is performed using a TosoHaas TSK-gei SW×1 column (4 mm×300 mm) and Agilent 1100 HPLC hardware including binary pump G1312A and RID detector G1362A plus Chemstation software A.08.03. This instrument can detect and quantitate hydrodynamic size of the cationic nucleic acid complexes and provide information on extinction coefficients of the nucleic acid component and molecular weight information of the complex through use of Zimm/Rouse equations for light scattering. A Brookhaven Instrument Corporation ZetaPALS dynamic light scattering instrument is used to measure size distributions of the cationic nucleic acid complexes and characterize the zeta-potential of these complexes.

### Example 10

### Formulation of Polycationic Complexes with Proteins, Peptides, or Antibodies

A protein, peptide, or antibody is complexed with a cationic compound based upon charge ratio or other properties such as hydrophobic interactions. The complex can be formulated with different charge ratios by using equivalents of protein, peptide, or antibody to cation to generate a formulation with a net positive charge (e.g. excess cation to protein, peptide, or antibody), a neutral charge, or a net negative charge (e.g. excess protein, peptide, or antibody to cation). The cation can be titrated into a solution of protein, peptide, or antibody or the protein, peptide, or antibody can be titrated into a solution of the cationic compound. In a non-limiting example, the protein, peptide, or antibody is obtained in HPLC purified form and dissolved in sterile Milli-Q water. A stock solution of cationic amine is added in a suitable amount based upon the number of anionic charges present in the protein, peptide, or antibody. For example, to generate a formulation with a net positive charge, an excess molar equivalence is used. The resulting solution is analyzed by strong anion exchange chromatography for concentration and purity. The solutions are then used in a cell culture assays to screen for efficacy of the formulated protein, peptide, or antibody composition in producing a therapeutic effect. Analytical techniques including size exclusion analysis and light scattering analysis as described herein can be used to further characterize the formulations.

### Example 11

### Formulation of Polycationic Complexes with Small Molecules

A small molecule (e.g. small molecule drug) is complexed with a cationic compound based upon charge ratio or other properties such as hydrophobic interactions. The complex can be formulated with different charge ratios by using equivalents of small molecule to cation to generate a formulation with a net positive charge (e.g. excess cation to the small molecule), a neutral charge, or a net negative charge (e.g. excess small molecule to cation). The cation can be titrated into a solution of the small molecule or the small molecule can be titrated into a solution of the cationic compound. In a non-limiting example, the small molecule is obtained in HPLC purified form and dissolved in sterile Milli-Q water. A stock solution of cationic amine is added in a suitable amount based upon the number of anionic charges present in the small molecule. For example, to generate a formulation with a net positive charge, an excess molar equivalence is used. The resulting solution is analyzed by strong anion exchange chromatography for concentration and purity. The solutions are then used in a cell culture assays to screen for efficacy of the formulated small molecule composition in producing a therapeutic effect. Analytical techniques including size exclusion analysis and light scattering analysis as described herein can be used to further characterize the formulations.

### Example 12

### Indications

The formulated siNA molecules of the invention can be used to treat a variety of diseases and conditions through modulation of gene expression. Using the methods described herein, formulated siNA molecules can be designed to modulate the expression any number of target genes, including but not limited to genes associated with cancer, metabolic diseases, infectious diseases such as viral, bacterial or fungal infections, neurologic diseases, musculoskeletal diseases, diseases of the immune system, diseases associated with signaling pathways and cellular messengers, and diseases associated with transport systems including molecular pumps and channels.

Non-limiting examples of various viral genes that can be targeted using formulated siNA molecules of the invention include Hepatitis C Virus (HCV, for example Genbank Accession Nos: D11168, D50483.1, L38318 and S82227), Hepatitis B Virus (HBV, for example GenBank Accession No. AF100308.1), Human Immunodeficiency Virus type 1 (HIV-1, for example GenBank Accession No. U51188), Human Immunodeficiency Virus type 2 (HIV-2, for example GenBank Accession No. X60667), West Nile Virus (WNV for example GenBank accession No. NC_{—}001563), cytomegalovirus (CMV for example GenBank Accession No. NC_{—}001347), respiratory syncytial virus (RSV for example GenBank Accession No. NC_{—}001781), influenza virus (for example example GenBank Accession No. AF037412, rhinovirus (for example, GenBank accession numbers: D00239, X02316, X01087, L24917, M16248, K02121, X01087), papillomavirus (for example GenBank Accession No. NC_{—}001353), Herpes Simplex Virus (HSV for example GenBank Accession No. NC_{—}001345), and other viruses such as HTLV (for example GenBank Accession No. AJ430458). Due to the high sequence variability of many viral genomes, selection of siNA molecules for broad therapeutic applications would likely involve the conserved regions of the viral genome. Non-limiting examples of conserved regions of the viral genomes include but are not limited to 5′-Non Coding Regions (NCR), 3′-Non Coding Regions (NCR) LTR regions and/or internal ribosome entry sites (IRES). siNA molecules designed against conserved regions of various viral genomes will enable efficient inhibition of viral replication in diverse patient populations and may ensure the effectiveness of the siNA molecules against viral quasi species which evolve due to mutations in the non-conserved regions of the viral genome.

Non-limiting examples of human genes that can be targeted using formulated siNA molecules of the invention using methods described herein include any human RNA sequence, for example those commonly referred to by Genbank Accession Number. These RNA sequences can be used to design siNA molecules that inhibit gene expression and therefore abrogate diseases, conditions, or infections associated with expression of those genes. Such non-limiting examples of human genes that can be targeted using siNA molecules of the invention include VEGFr (VEGFR1 for example GenBank Accession No. XM_{—}067723, VEGFR2 for example GenBank Accession No. AF063658), HER1, HER2, HER3, and HER4 (for example Genbank Accession Nos: NM_{—}005228, NM_{—}004448, NM_{—}001982, and NM_{—}005235 respectively), telomerase (TERT, for example GenBank Accession No. NM_{—}003219), telomerase RNA (for example GenBank Accession No. U86046), NFkappaB, Rel-A (for example GenBank Accession No. NM_{—}005228), NOGO (for example GenBank Accession No. AB020693), NOGOr (for example GenBank Accession No. XM_{—}015620), RAS (for example GenBank Accession No. NM 004283), RAF (for example GenBank Accession No. XM_{—}033884), CD20 (for example GenBank Accession No. X07203), METAP2 (for example GenBank Accession No. NM_{—}003219), CLCA1 (for example GenBank Accession No. NM_{—}001285), phospholamban (for example GenBank Accession No. NM_{—}002667), PTPIB (for example GenBank Accession No. M31724), PCNA (for example GenBank Accession No. NM_{—}002592.1), PKC-alpha (for example GenBank Accession No. NM_{—}002737) and others. The genes described herein are provided as non-limiting examples of genes that can be targeted using siNA molecules of the invention. Additional examples of such genes are described by accession number in Beigelman et al., U.S. Ser. No. 60/363,124, filed Mar. 11, 2002 and incorporated by reference herein in its entirety.

One skilled in the art would readily appreciate that the present invention is well adapted to carry out the objects and obtain the ends and advantages mentioned, as well as those inherent therein. The methods and compositions described herein are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by various embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Other embodiments are within the following claims.

TABLE I

| | | | | | |
|---|---|---|---|---|---|
| | | | | Wait Time* 2′-O- | |
| Reagent | Equivalents | Amount | Wait Time* DNA | methyl | Wait Time* RNA |

A. 2.5 μmol Synthesis Cycle ABI 394 Instrument

| | | | | | | | | | |
|---|---|---|---|---|---|---|---|---|---|
| Phosphoramidites | 6.5 | 163 | μL | 45 | sec | 2.5 | min | 7.5 | min |
| S-Ethyl Tetrazole | 23.8 | 238 | μL | 45 | sec | 2.5 | min | 7.5 | min |
| Acetic Anhydride | 100 | 233 | μL | 5 | sec | 5 | sec | 5 | sec |
| N-Methyl | 186 | 233 | μL | 5 | sec | 5 | sec | 5 | sec |
| Imidazole | | | | | | | | | |
| TCA | 176 | 2.3 | mL | 21 | sec | 21 | sec | 21 | sec |
| Iodine | 11.2 | 1.7 | mL | 45 | sec | 45 | sec | 45 | sec |
| Beaucage | 12.9 | 645 | μL | 100 | sec | 300 | sec | 300 | sec |

| | | | | | | |
|---|---|---|---|---|---|---|
| Acetonitrile | NA | 6.67 | mL | NA | NA | NA |

B. 0.2 μmol Synthesis Cycle ABI 394 Instrument

| | | | | | | | | | |
|---|---|---|---|---|---|---|---|---|---|
| Phosphoramidites | 15 | 31 | μL | 45 | sec | 233 | sec | 465 | sec |
| S-Ethyl Tetrazole | 38.7 | 31 | μL | 45 | sec | 233 | min | 465 | sec |
| Acetic Anhydride | 655 | 124 | μL | 5 | sec | 5 | sec | 5 | sec |
| N-Methyl | 1245 | 124 | μL | 5 | sec | 5 | sec | 5 | sec |
| Imidazole | | | | | | | | | |
| TCA | 700 | 732 | μL | 10 | sec | 10 | sec | 10 | sec |
| Iodine | 20.6 | 244 | μL | 15 | sec | 15 | sec | 15 | sec |
| Beaucage | 7.7 | 232 | μL | 100 | sec | 300 | sec | 300 | sec |

| | | | | | | |
|---|---|---|---|---|---|---|
| Acetonitrile | NA | 2.64 | mL | NA | NA | NA |

C. 0.2 μmol Synthesis Cycle 96 well Instrument

| | Equivalents: DNA/ | Amount: DNA/2′-O- | | | | Wait Time* 2′-O- | | | |
|---|---|---|---|---|---|---|---|---|---|
| Reagent | 2′-O-methyl/Ribo | methyl/Ribo | | Wait Time* DNA | | methyl | | Wait Time* Ribo | |
| Phosphoramidites | 22/33/66 | 40/60/120 | μL | 60 | sec | 180 | sec | 360 | sec |
| S-Ethyl Tetrazole | 70/105/210 | 40/60/120 | μL | 60 | sec | 180 | min | 360 | sec |
| Acetic Anhydride | 265/265/265 | 50/50/50 | μL | 10 | sec | 10 | sec | 10 | sec |
| N-Methyl | 502/502/502 | 50/50/50 | μL | 10 | sec | 10 | sec | 10 | sec |
| Imidazole | | | | | | | | | |
| TCA | 238/475/475 | 250/500/500 | μL | 15 | sec | 15 | sec | 15 | sec |
| Iodine | 6.8/6.8/6.8 | 80/80/80 | μL | 30 | sec | 30 | sec | 30 | sec |
| Beaucage | 34/51/51 | 80/120/120 | | 100 | sec | 200 | sec | 200 | sec |

| | | | | | | |
|---|---|---|---|---|---|---|
| Acetonitrile | NA | 1150/1150/1150 | μL | NA | NA | NA |

*Wait time does not include contact time during delivery.

TABLE II
Non-limiting examples of Stabilization Chemistries for chemically
modified siNA constructs

| Chemistry | pyrimidine | Purine | cap | p = S | Strand |
|---|---|---|---|---|---|
| “Stab 00” | Ribo | Ribo | TT at 3′- | | S/AS |
| | | | ends | | |
| “Stab 1” | Ribo | Ribo | — | 5 at 5′-end | S/AS |
| | | | | 1 at 3′-end | |
| “Stab 2” | Ribo | Ribo | — | All | Usually AS |
| | | | | linkages | |
| “Stab 3” | 2′-fluoro | Ribo | — | 4 at 5′-end | Usually S |
| | | | | 4 at 3′-end | |
| “Stab 4” | 2′-fluoro | Ribo | 5′ and 3′- | — | Usually S |
| | | | ends | | |
| “Stab 5” | 2′-fluoro | Ribo | — | 1 at 3′-end | Usually AS |
| “Stab 6” | 2′-O- | Ribo | 5′ and 3′- | — | Usually S |
| | Methyl | | ends | | |
| “Stab 7” | 2′-fluoro | 2′-deoxy | 5′ and 3′- | — | Usually S |
| | | | ends | | |
| “Stab 8” | 2′-fluoro | 2′-O- | — | 1 at 3′-end | Usually AS |
| | | Methyl | | | |
| “Stab 9” | Ribo | Ribo | 5′ and 3′- | — | Usually S |
| | | | ends | | |
| “Stab 10” | Ribo | Ribo | — | 1 at 3′-end | Usually AS |
| “Stab 11” | 2′-fluoro | 2′-deoxy | — | 1 at 3′-end | Usually AS |
| “Stab 12” | 2′-fluoro | LNA | 5′ and 3′- | | Usually S |
| | | | ends | | |
| “Stab 13” | 2′-fluoro | LNA | | 1 at 3′-end | Usually AS |
| “Stab 14” | 2′-fluoro | 2′-deoxy | | 2 at 5′-end | Usually AS |
| | | | | 1 at 3′-end | |
| “Stab 15” | 2′-deoxy | 2′-deoxy | | 2 at 5′-end | Usually AS |
| | | | | 1 at 3′-end | |
| “Stab 16” | Ribo | 2′-O- | 5′ and 3′- | | Usually S |
| | | Methyl | ends | | |
| “Stab 17” | 2′-O- | 2′-O- | 5′ and 3′- | | Usually S |
| | Methyl | Methyl | ends | | |
| “Stab 18” | 2′-fluoro | 2′-O- | 5′ and 3′- | | Usually S |
| | | Methyl | ends | | |
| “Stab 19” | 2′-fluoro | 2′-O- | 3′-end | | Usually AS |
| | | Methyl | | | |
| “Stab 20” | 2′-fluoro | 2′-deoxy | 3′-end | | Usually AS |
| “Stab 21” | 2′-fluoro | Ribo | 3′-end | | Usually AS |
| “Stab 22” | Ribo | Ribo | 3′-end — | | Usually AS |
| “Stab 23” | 2′-fluoro* | 2′- | 5′ and 3′- | | Usually S |
| | | deoxy* | ends | | |
| “Stab 24” | 2′-fluoro* | 2′-O- | — | 1 at 3′-end | Usually AS |
| | | Methyl* | | | |
| “Stab 25” | 2′-fluoro* | 2′-O- | — | 1 at 3′-end | Usually AS |
| | | Methyl* | | | |
| “Stab 26” | 2′-fluoro* | 2′-O- | — | | Usually AS |
| | | Methyl* | | | |

CAP = any terminal cap, see for example FIG. 10.
All Stab 00-26 chemistries can comprise 3′-terminal thymidine (TT) residues
All Stab 00-26 chemistries typically comprise about 21 nucleotides, but can vary as described herein.
S = sense strand
AS = antisense strand
*Stab 23 has a single ribonucleotide adjacent to 3′-CAP
*Stab 24 has asingle ribonucleotide at 5′-terminus
*Stab 25 and Stab 26 have three ribonucleotides at 5′-terminus
p = phosphorothioate linkage

1. A composition comprising a short interfering nucleic acid (siNA) molecule and a compound having the Formula 1: 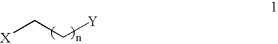
wherein X and Y are the same or different and represent an amine, substituted amine, guanidinium, substituted guanidinium, histidyl, or substituted histidyl group, and n represents an integer from 0 to about 20.

2. A composition comprising a short interfering nucleic acid (siNA) molecule and a compound having the Formula 2: 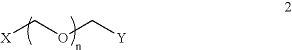
wherein X and Y are the same or different and represent an amine, substituted amine, guanidinium, substituted guanidinium, histidyl, or substituted histidyl group, and n represents an integer from about 1 to about 20.

3. A composition comprising a short interfering nucleic acid (siNA) molecule and a compound having the Formula 3: 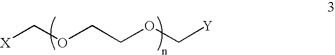
wherein X and Y are the same or different and represent an amine, substituted amine, guanidinium, substituted guanidinium, histidyl, or substituted histidyl group, and n represents an integer from about 1 to about 20.

4. A composition comprising a short interfering nucleic acid (siNA) molecule and a compound having the Formula 4: 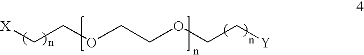
wherein X and Y are the same or different and represent an amine, substituted amine, guanidinium, substituted guanidinium, histidyl, or substituted histidyl group and each n independently represents an integer from about 1 to about 20.

5. A composition comprising a short interfering nucleic acid (siNA) molecule and a compound having the Formula 5: 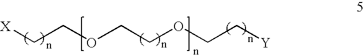
wherein X and Y are the same or different and represent an amine, substituted amine, guanidinium, substituted guanidinium, histidyl, or substituted histidyl group, and each n independently represents an integer from about 1 to about 20.

6. A composition comprising a short interfering nucleic acid (siNA) molecule and a compound having the Formula 6: 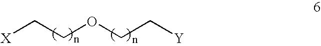
wherein X and Y are the same or different and represent an amine, substituted amine, guanidinium, substituted guanidinium, histidyl, or substituted histidyl group, and each n independently represents an integer from 0 to about 20.

7. A composition comprising a short interfering nucleic acid (siNA) molecule and a compound having the Formula 7: 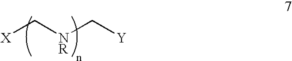
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and n represents an integer from about 1 to about 20.

8. A composition comprising a short interfering nucleic acid (siNA) molecule and a compound having the Formula 8: 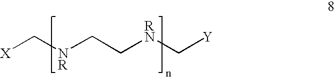
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and n represents an integer from about 1 to about 20.

9. A composition comprising a short interfering nucleic acid (siNA) molecule and a compound having the Formula 9: 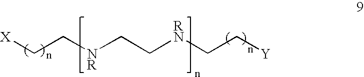
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and each n independently represents an integer from about 1 to about 20.

10. A composition comprising a short interfering nucleic acid (siNA) molecule and a compound having the Formula 10: 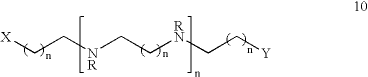
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and each n independently represents an integer from about 1 to about 20.

11. A composition comprising a short interfering nucleic acid (siNA) molecule and a compound having the Formula 11: 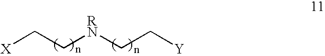
wherein X and Y are the same or different and represent an amine, substituted amine, guanidinium, substituted guanidinium, histidyl, or substituted histidyl group that can be the same or different, R represents H, alkyl, substituted alkyl, aryl, substituted aryl, or a ligand, and each n independently represents an integer from 0 to about 20.

12. A composition comprising a short interfering nucleic acid (siNA) molecule and a compound having the Formula 12: 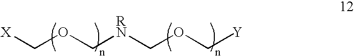
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and each n independently represents an integer from 0 to about 20.

13. A composition comprising a short interfering nucleic acid (siNA) molecule and a compound having the Formula 13: 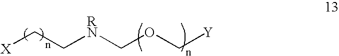
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, and each n independently represents an integer from 0 to about 20.

14. A composition comprising a short interfering nucleic acid (siNA) molecule and a compound having the Formula 14: 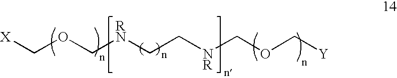
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and n′ represents an integer from about 1 to about 20.

15. A composition comprising a short interfering nucleic acid (siNA) molecule and a compound having the Formula 15: 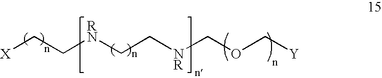
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and n′ represents an integer from about 1 to about 20.

16. A composition comprising a short interfering nucleic acid (siNA) molecule and a compound having the Formula 16: 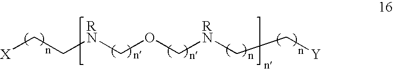
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and each n′ independently represents an integer from about 1 to about 10.

17. A composition comprising a short interfering nucleic acid (siNA) molecule and a compound having the Formula 17: 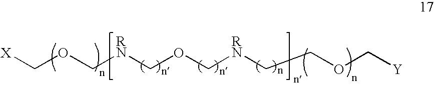
wherein X and Y are the same or different and represent an amine, substituted amine, guanidinium, substituted guanidinium, histidyl, or substituted histidyl group R represents H, alkyl, substituted alkyl, aryl, substituted aryl, or a ligand, each n independently represents an integer from 0 to about 20, and each n′ independently represents an integer from about 1 to about 10.

18. A composition comprising a short interfering nucleic acid (siNA) molecule and a compound having the Formula 18: 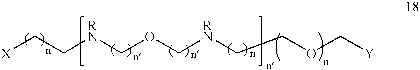
wherein X and Y are the same or different and represent an amine, substituted amine, guanidinium, substituted guanidinium, histidyl, or substituted histidyl group, R represents H, alkyl, substituted alkyl, aryl, substituted aryl, or a ligand, each n independently represents an integer from 0 to about 20, and each n′ independently represents an integer from about 1 to about 10.

19. A composition comprising a short interfering nucleic acid (siNA) molecule and a compound having the Formula 19: 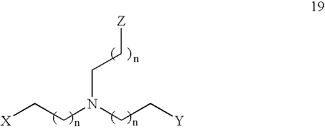
wherein X, Y and Z are the same or different and represent an amine, substituted amine, guanidinium, substituted guanidinium, histidyl, or substituted histidyl, and each n independently represents an integer from about 1 to about 20.


SEQUENCE LISTING (6 sequences)

SEQ ID NO: 1 - RNA, 21 nt, Artificial Sequence
  synthetic
ugugcacuucgcuucaccuuu

SEQ ID NO: 2 - DNA, 21 bp, Artificial Sequence
  synthetic
aggugaagcgaagugcacatt

SEQ ID NO: 3 - DNA, 21 bp, Artificial Sequence
  synthetic
ugugcacuucgcuucaccutt

SEQ ID NO: 4 - DNA, 21 bp, Artificial Sequence
  synthetic
aggugaagcgaagugcacatt

SEQ ID NO: 5 - DNA, 21 bp, Artificial Sequence
  synthetic
uccacuucgcuucacgugutt

SEQ ID NO: 6 - DNA, 21 bp, Artificial Sequence
  synthetic
acacgugaagcgaaguggatt